(19) United States
(12) Patent Application Publication
(10) Pub. No.: US 2025/0248919 A1
(43) Pub. Date: Aug. 7, 2025

(54) NON-SOLID COSMETIC COMPOSITION WITH AN APOLAR VOLATILE HYDROCARBON OIL, A SOLVENT WITH AN ALCOHOL, ACID, AMINE OR AMIDE FUNCTION AND A SILICONE ELASTOMER WITH CARBOXYLIC ACID FUNCTIONS

(71) Applicant: L'OREAL, Paris (FR)

(72) Inventors: Guillaume KERGOSIEN, Chevilly Larue (FR); Simon COLLET, Chevilly Larue (FR); Kévin DU, Chevilly Larue (FR); Mary Kay CROFOOT, Auburn, MI (US); Roxanne HALLER, Auburn, MI (US)

(21) Appl. No.: 18/434,567

(22) Filed: Feb. 6, 2024

(51) Int. Cl. A61K 8/898; A61Q 1/00; A61Q 19/00

(52) U.S. Cl. CPC A61K 8/898; A61Q 1/00; A61Q 19/00; A61K 2800/594

(57) ABSTRACT

The present invention relates to a non-solid composition for caring for and/or making up keratinous materials, in particular the skin, comprising, preferably in a physiologically acceptable medium:
a) an oily phase comprising
i) at least one apolar volatile hydrocarbon oil H₁ and
ii) at least one liquid hydrocarbon solvent with a molecular mass of less than 600 g/mol comprising at least one alcohol, carboxylic acid, amine or amide function and mixtures thereof; with the proviso that when the liquid hydrocarbon solvent is a primary alcohol, it comprises at least 3 carbon atoms, and
b) at least one carboxylic acid functional silicone elastomer;
the ratio by weight of the total amount of volatile oil(s) to the total amount of hydrocarbon liquid solvent(s) ii) being less than or equal to 20.0.

The invention also relates to a process for coating keratinous materials, more particularly for making up and/or caring for keratinous materials, such as the skin, characterized in that it comprises the application to the keratinous materials of a composition as defined above.

### TECHNICAL AREA

The present invention aims to provide, for the field of care and/or makeup for keratin materials, in particular the skin, a new non-solid composition comprising a) an oily phase comprising i) at least one apolar volatile hydrocarbon-based oil and ii) at least one liquid hydrocarbon solvent comprising at least one alcohol, acid, amine or amide function and b) at least one silicone elastomer containing carboxylic acid functions.

Cosmetic make-up compositions are commonly used to provide an aesthetic color to keratinous materials such as the skin, but also to beautify uneven skin by making it possible to hide spots and dyschromias, to reduce the visibility of relief imperfections such as pores and wrinkles as well as pimples and traces of acne.

They are also looking for care and/or makeup products for keratin materials, in particular for the skin, which have a comfortable texture at the sensory level and on application, in particular which are easily removed and which spread easily over the area of keratin material to be covered.

Makeup compositions based on carboxylic acid-functional silicone elastomers with good performance in terms of staying power and durability have already been proposed in published documents WO2015066161 (US20160200876), WO2015066165, WO2015066199, WO2015167963, WO20180165434 (U.S. Pat. No. 10,918,587B2), IPCOM000250448D, IPCOM000250657D. However, these polymers generally have a texture that is difficult to pick up and apply and tend to result in makeup compositions whose consistency does not allow the product to be easily picked up and spread on the skin.

One of the objectives of this invention is to realize a care and/or make-up composition comprising at least one silicone elastomer with carboxylic acid functions which is stable, and having a suitable, flexible and homogeneous consistency for being taken up and easily spread on the keratinous material to be covered, in particular the skin.

In the course of her research, the applicant unexpectedly discovered that this objective could be achieved with a non-solid composition for caring for and/or making up keratinous materials, in particular the skin, comprising, preferably in a physiologically acceptable medium:
a) an oily phase comprising
i) at least one apolar volatile hydrocarbon oil H₁ and
ii) at least one liquid hydrocarbon solvent with a molecular mass of less than 600 g/mol comprising at least one alcohol, carboxylic acid, amine or amide function and mixtures thereof; with the proviso that when the liquid hydrocarbon solvent is a primary alcohol, it comprises at least 3 carbon atoms, an
b) at least one carboxylic acid functional silicone elastomer;
the ratio by weight of the total amount of volatile oil(s) to the total amount of hydrocarbon liquid solvent(s) ii) being less than or equal to 20.0.

This discovery is the basis of the invention.

The present invention relates to a non-solid composition for the care and/or make-up of keratinous materials, in particular the skin, comprising, preferably in a physiologically acceptable medium:
a) an oily phase comprising
i) at least one apolar volatile hydrocarbon oil H₁ and
ii) at least one liquid hydrocarbon solvent with a molecular mass of less than 600 g/mol comprising at least one alcohol, carboxylic acid, amine or amide function and mixtures thereof; with the proviso that when the liquid hydrocarbon solvent is a primary alcohol, it comprises at least 3 carbon atoms, and
b) at least one carboxylic acid functional silicone elastomer;
the ratio by weight of the total amount of volatile oil(s) to the total amount of hydrocarbon liquid solvent(s) ii) being less than or equal to 20.0.

The invention also relates to a process for coating keratinous materials, more particularly for making up and/or caring for keratinous materials, such as the skin, characterized in that it comprises the application to the keratinous materials of a composition as defined above.

The invention relates more particularly to a method of making up and/or caring for the skin, characterized in that it comprises the application to the skin of a composition as defined above.

### DEFINITIONS

In the context of the present invention, by “keratinous materials” is meant the skin and more particularly areas such as the face, the cheeks, the hands, the body, the legs and thighs, the eye contour, the eyelids.

“Physiologically acceptable” is understood to mean compatible with the skin and/or its appendages, which has a pleasant color, odor and touch and which does not generate unacceptable discomfort (tingling, tightness) likely to deter the consumer from using this composition.

By “non-solid composition” is meant any composition whose hardness is less than 10 Newtons, preferably less than 5 Newtons, preferably less than 2 Newtons, more preferably less than 1 Newton and most preferably less than 0.5 Newton, assessed under the measurement conditions specified as follows.

The hardness measurements are carried out on the formulas obtained after manufacture, as is (finished products without packaging). The hardness of the formulas is measured after 24 hours of waiting at 25° C. and 40% of hygrometry. The jars containing the samples are characterized by texturometry using a texturometer such as TA.XTplus Texture Analyzer® marketed by the company Stable Micro Systems, according to the following protocol:

The hardness corresponds to the force required, in Newton, for the punch to penetrate the composition by 2 mm. Depending on the shape of the products, the tests are repeated between 2 and 5 times per product. A P/20 type punch (20 mm diameter) is brought into contact with the sample at a speed of 1 mm/s. The measuring system detects the interface with the sample with a detection threshold equal to 0.02 Newton. The probe is inserted 2 mm into the sample at a speed of 1 mm/s. The measuring device records the evolution of the force measured in compression over time, during the penetration phase. The hardness of the sample corresponds to the average of the maximum values of the force detected during the penetration, over 2 to 5 measurements.

### Oily Phase

The composition according to the invention for caring for and/or making up keratinous materials comprises at least one oily phase comprising
i) at least one apolar volatile hydrocarbon oil H₁ and
ii) at least one liquid hydrocarbon solvent with a molecular mass of less than 600 g/mol comprising at least one alcohol, carboxylic acid, amine or amide function and mixtures thereof; with the proviso that, when the liquid hydrocarbon solvent is a primary alcohol, it comprises at least 3 carbon atoms.

By “oil” is meant a fatty substance which is liquid at room temperature (25° C.) and atmospheric pressure (760 mm Hg or 105 Pa).

“Oily phase” is understood to mean a liquid organic phase at room temperature (25° C.) and atmospheric pressure containing at least one oil and possibly cosmetic additives which are soluble or miscible in the said phase.

“Apolar hydrocarbon oil” is understood to mean an oil containing only hydrogen and carbon atoms.

By “volatile oil” is meant, within the meaning of the invention, any oil capable of evaporating on contact with the skin in less than one hour, at room temperature and atmospheric pressure. The volatile oil is a volatile cosmetic compound which is liquid at room temperature, having in particular a non-zero vapour pressure at room temperature and atmospheric pressure, in particular having a vapour pressure ranging from 2.66 Pa to 40,000 Pa, in particular ranging from 2.66 Pa to 13,000 Pa, and more particularly ranging from 2.66 Pa to 1300 Pa.

### Apolar Volatile Hydrocarbon Oil H₁

As an example of an apolar volatile hydrocarbon oil H₁ which can be used in the oil phase of the composition of the invention, mention may be made of C8-C16 isoalkanes of petroleum origin (also called isoparaffins) such as isododecane (also called 2,2,4,4,6-pentamethylheptane), isodecane and isohexadecane.

The apolar volatile hydrocarbon oils H₁ usable in the compositions according to the invention may be selected from volatile linear alkanes comprising from 9 to 14 carbon atoms.

As examples of linear C9-C14 alkanes suitable for the invention, mention may be made of the alkanes described in the patent applications of the Cognis company WO 2007/068371, or WO2008/155059 (mixtures of distinct alkanes differing by at least one carbon). These alkanes are obtained from fatty alcohols, themselves obtained from copra or palm oil.

As examples of linear C9-C14 alkanes suitable for the invention, mention may be made of n-nonane (C9), n-decane (C10), n-undecane (C11), n-dodecane (C12), n-tridecane (C13), n-tetradecane (C14), and mixtures thereof.

Particular mention may be made of n-dodecane (C12) and n-tetradecane (C14) sold by Sasol respectively under the references PARAFOL 12 97® and PARAFOL 14 97®, as well as their mixtures.

According to another embodiment, a mixture of n-dodecane and n-tetradecane is used. In particular, the mixture of dodecane/tetradecane in the weight ratio 85/15 marketed by the company BIOSYNTHIS under the reference VEGELIGHT 1214® may be used.

According to yet another embodiment, a mixture of volatile linear C9-C12 alkanes with the INCI name: C9-12 ALKANE such as the product marketed by the company BIOSYNTHIS under the reference VEGELIGHT SILK® is used.

[According to yet another mode of realization, a mixture of n-undecane (C11) and n-tridecane (C13) such as those obtained in Examples 1 and 2 of application WO2008/155059 of the company Cognis and such as that sold under the trade name CETIOL ULTIMATE® by the company BASF is used.

According to a particularly preferred mode, the apolar volatile hydrocarbon oil H1 will be chosen from C8-C16 isoalkanes of petroleum origin, and more particularly isododecane.

According to a preferred mode of the invention, the apolar volatile hydrocarbon oil(s) H₁ is (are) present in contents ranging from 50 to 90% by weight, and more preferably ranging from 70 to 90% by weight relative to the total weight of the oil phase.

According to a preferred mode of the invention, the apolar volatile hydrocarbon oil(s) H₁ is (are) present in contents ranging from 45 to 85% by weight, and more preferably ranging from 55 to 75% by weight with respect to the total weight of the composition.

Liquid Hydrocarbon Solvent ii) with Alcohol, Carboxylic Acid, Amine or Amide Function

According to the invention, the oily phase of the composition comprises at least one hydrocarbon liquid solvent comprising at least one alcohol, carboxylic acid, amine and/or amide function and having a molecular mass of less than 600 g/mol, with the proviso that when the hydrocarbon liquid solvent is a primary alcohol, it comprises at least 3 carbon atoms.

Preferably, the molecular weight is less than 500 g/mol, more preferably less than 400 g/mol, and more particularly less than 300 g/mol.

By “hydrocarbon solvent”, is meant any organic solvent containing hydrogen atoms, carbon atoms and at least one alcohol, carboxylic acid, amine and/or amide function.

By “non-volatile solvent” is meant an organic solvent remaining on the keratinous material at room temperature (25° C.) and atmospheric pressure (760 mm Hg) for at least several hours and having in particular a vapour pressure of less than 10⁻³ mm Hg (0.13 Pa).

By “primary alcohol” is meant an alcohol in which the hydroxyl group —OH is carried by a primary carbon atom, i.e. bonded to a single other carbon atom. A primary alcohol has the structure R—CH₂—OH:

Among the liquid hydrocarbon solvents ii) comprising at least one alcohol function usable in the composition of the invention, ethanol comprising 2 carbon atoms may be mentioned.

According to a particular mode, the hydrocarbon liquid solvent is ethanol.

According to a particular mode, the liquid hydrocarbon solvent is chosen from primary alcohols comprising at least 3 carbon atoms, with a molecular weight of less than 500 g/mol, preferably 400 g/mol, and more preferably less than 300 g/mol. We can mention, for example:
isopropanol (INCI name: Isopropyl Alcohol) such as the commercial product sold under the NAME EXXONMOBIL™ IPA PHARMA® by the company ExxonMobil Chemical Europe
n-hexanol such as the commercial products ALFOL 6 ALCOHOL® from Sasol North America, Inc. and NACOL 6-99 ALCOHOL from Sasol Germany GmbH; Hamburg);
1-nonanol available from Sigma Aldrich;
octyldodecanol such as the commercial products AEC OCTYLDODECANOL® (A & E Connock Perfumery & Cosmetics Ltd.), EUTANOL G® (BASF Corporation), EXXAL 20® (ExxonMobil Chemical Company), ISOFOL 20 ALCOHOL® (Sasol Germany GmbH Hamburg), JARCOL I-20® (Jarchem Industries), MICHEL XO-150-20 ISO-EICOSYL ALCOHOL® (M. Michel and Company, Inc.), RISONOL 20SP® (Kokyu Alcool Kogyo Co., Ltd.), SABODERM G20® (Sabo s.p.a.), U-TANOL G® (Universal Preserv-A-Chem, Inc.);
2-hexyldecanol such as the commercial products EUTANOL G 16® (BASF Corporation), EXXAL 16® (ExxonMobil Chemical Company), FINEOXOCOL 1600K® (Nissan Chemical Industries), ISOFOL 16 ALCOHOL® (Sasol Germany GmbH Hamburg), JARCOL I-16® (Jarchem Industries), RISONOL 16SP® (Kokyu Alcohol Kogyo Co., Ltd;
oleic alcohol (INCI name Oleyl Alcohol) such as the commercial products AEC OLEYL ALCOHOL® (A & E Connock (Perfumery & Cosmetics) Ltd.), HD-EUTANOL V® (BASF Corporation), JEECOL O® (Jeen International Corporation), LIPOCOL O/95® (Vantage Personal Care), NOVOL (Croda, Inc.), OLEYL ALCOHOL VP® (Kokyu Alcohol Kogyo Co., Ltd.), ORISTAR OAH (Orient Stars LLC), SABONAL 90/95 (Sabo s.p.a.), U-TANOL HD 80/85®, U-TANOL HD 90/95® and U-TANOL HD CG® (Universal Preserv-A-Chem, Inc.);
isostearyl alcohol (INCI name: Isostearyl Alcohol) such as the commercial products AEC ISOSTEARYL ALCOHOL® (A & E Connock (Perfumery & Cosmetics) Ltd.), FINEOXOCOL 180® (Nissan Chemical Industries), ISOSTEARYL ALCOHOL EX® (Kokyu Alcohol Kogyo Co., Ltd.), JARCOL 1-18 CG® and JARCOL I-18T® (Jarchem Industries), JEECOL ISA® (Jeen International Corporation), MICHEL XO-150-1620 ISO-STEARYL ALCOHOL® (M. Michel and Company, Inc.), PRISORINE 3515® (Croda Europe, Ltd.), RISONOL 18SP® (Kokyu Alcohol Kogyo Co., Ltd.);
2-butyloctanol such as the commercial products ISOFOL 12 ALCOHOL® (Sasol Germany GmbH Hamburg), JARCOL I-12® (Jarchem Industries), MICHEL XO-150-12 ISO-LAURYL ALCOHOL® (M. Michel and Company, Inc.);
undecylpentanedecanol such as the commercial product EXXAL 26® (ExxonMobil Chemical Company).

Among these primary alcohol solvents, those in the group comprising isopropanol, octyldodecanol, n-hexanol, 1-nonanol, 2-hexyldecanol, oleic alcohol, and mixtures thereof will be chosen more particularly.

Among liquid hydrocarbon solvents ii) comprising at least one alcohol function that can be used in the composition of the invention, mention may also be made of secondary alcohols with the structure such as esters of lactic acid and of C12-C15 monoalcohol, such as for example:
lauryl lactate such as the commercial products AEC LAURYL LACTATE® (A & E Connock Perfumery & Cosmetics) Ltd.), CERAPHYL 31® (Ashland Inc.), CETINOL LL® (Fabriquimica S.R.L.), DERMOL LL® (Alzo International Inc.), EXCEPARL LM-LC® (Kao Chemicals Europe S.L.), PELEMOL LL® (Phoenix Chemical, Inc.), SCHERCEMOL LL ESTER® (Lubrizol Advanced Materials, Inc.)
myristyl lactate such as the commercial products AEC MYRISTYL LACTATE® (A & E Connock Perfumery & Cosmetics) Ltd.), CETINOL LM® (Fabriquimica S.R.L.), CRODAMOL ML® (Croda Europe, Ltd.), DERMOL ML® (Alzo International Inc.), DUB LM® (Stearinerie Dubois Fils), JEECHEM ML® (Jeen International Corporation), LACTABASE C14® (Laboratoires Prod'Hyg), NIKKOL MYRISTYL LACTATE® (Nikko Chemicals Co., Ltd.), PELEMOL ML® (Phoenix Chemical, Inc.), SABODERM MLC® (Sabo s.p.a.), UNIESTER LM® (Chemyunion Quimica Ltd.);
C12-C13 alkyl lactate such as the commercial product COSMACOL ELI® (Sasol Italy S.p.A.);
C12-C15 alkyl lactate such as the commercial products CERAPHYL 41® (Ashland Inc.), DERMOL® (Alzo International Inc.), DUB L1215® (Stearinerie Dubois Fils). C12-C13 alkyl lactate will be used more preferably.

Among the liquid hydrocarbon solvents ii) comprising at least one carboxylic acid function which can be used in the composition of the invention, mention may be made of unsaturated fatty acids with a molecular mass of less than 600 g/mol such as:
oleic acid such as the commercial products linoleic acid AEC OLEIC ACID (A & E Connock Perfumery & Cosmetics) Ltd.), EMERSOL 213,® EMERSOL 221® EMERSOL 6321® and EMERSOL 233LL® (Emery Oleochemicals LLC), ORISTAR OLA® (Orient Stars LLC), RADIACID 0212-0216® (Oleon NV), UNIFAT 5L® (Universal Preserv-A-Chem, Inc.),
linoleic acid such as the commercial products ACIDE LINOLEIQUE PURETE®≥70%, ACIDE LINOLEIQUE PURETE®≥98% (Phytocos Laboratories), EMERSOL 315® (Emery Oleochemicals LLC), OriStar CLA® and OriStar LA® (Orient Stars LLC®).
dilinoeic acid such as the commercial product UNIDYME 14® (Arizona Chemical Company, LLC);
linolenic acid such as the commercial products ABS ACAI STEROLS EFA® (Active Concepts LLC), DUB OMEGA® and DUB OMEGA 6 HYDRO® (Stearinerie Dubois Fils), EFADERMA® and EFADERMASTEROLO® (Vevy Europe SpA), LIANT TW 876 ® (Sensient Cosmetic Technologies), LIPOTROPHYNE-A® (Vevy Europe SpA), VITAMIN F FORTE A® and VITAMIN F WATER-SOLUBLE CLR® (CLR), VITAMIN F OILSOLUBLE N and VITAMIN F WATERSOLUBLE N® (Lipoid Kosmetik AG), and their mixtures.

Among the liquid hydrocarbon solvents ii) comprising at least one amine function which can be used in the composition of the invention, mention may be made of secondary dialkylamines (HNR1R2) and tertiary trialkyamines (NR1R2R3) with a molecular mass of less than 600 g/mol as:
dioctylamine (INCI name Diethylhexylamine) available from Sigma Aldrich
trioctylamine available from Sigma Aldrich, and mixtures thereof.

Among the liquid hydrocarbon solvents comprising at least one amide function which can be used in the composition of the invention, mention may be made of
Isopropyl Lauroyl Sarcosinate of structure

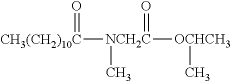

such as the product sold under the trade name ELDEW SL-205® by the company Ajinomoto Health & Nutrition North America, Inc;
N-acetyl N-butylaminopropionate ‘(INCI name: Ethyl Butylacetylaminopropionate) of structure

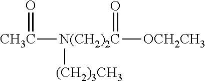

such as the product sold under the trade name IR3535® by the company MERCK;
the N,N-Dimethyldecanamide of structure

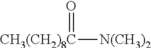

marketed under the name SPECTRASOLV DMDA® by The Hallstar Company;
N,N-Dimethylcaprylamide or N,N-Dimethyloctanamide sold under the name HALLCOMID® M-8-10 by the company STEPAN, and mixtures thereof. More particularly, Isopropyl Lauroyl Sarcosinate will be used.

According to a preferred embodiment of the invention, the ratio by weight of the total quantity of volatile oil(s) relative to the total weight of the composition to the total quantity of liquid hydrocarbon solvent(s) (s) ii) based on the total weight of the composition ranges from 5.0 to 20.0.

According to a preferred embodiment of the invention, the liquid hydrocarbon solvent(s) with an alcohol, carboxylic acid, amine or amide function is (are) present at levels ranging from 1 to 20% by weight, and more preferably ranging from 2 to 15% by weight relative to the total weight of the composition.

### Carboxylic Acid Functional Silicone Elastomer

The composition according to the invention for caring for and/or making up keratin materials comprises b) at least one silicone elastomer containing carboxylic acid functions.

By “organopolysiloxane elastomer” is meant a flexible, deformable organopolysiloxane having viscoelastic properties and in particular the consistency of a sponge or a flexible sphere. Its modulus of elasticity is such that this material resists deformation and has a limited capacity for extension and contraction. This material is able to regain its original shape after stretching. In general is partially or totally cross-linked and is non-cyclic.

The term “silicone elastomer with carboxylic acid functions” means an organopolysiloxane elastomer comprising in its structure at least two carboxylic acid groups with at least one —COOH function.

The composition according to the invention advantageously has a content of active material of silicone elastomer with carboxylic acid functions ranging from 2 to 25% by weight relative to the weight of the composition. Preferably, this content ranges from 5 to 20% by weight relative to the weight of the composition.

The carboxylic acid-functional silicone elastomers usable in the compositions of the invention may be chosen from those described in the published documents WO2015066161 (US20160200876), WO2015066165, WO2015066199, WO2015167963, WO20180165434 (U.S. Pat. No. 10,918,587B2), IPCOM000250448D, IPCOM000250657D.

According to a particular mode, the carboxylic acid functional silicone elastomer is selected from those having the following formula (I):

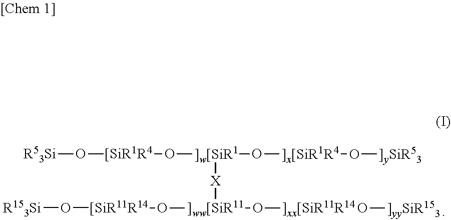

in which the radical X corresponds to the following formula (i) or (i′):

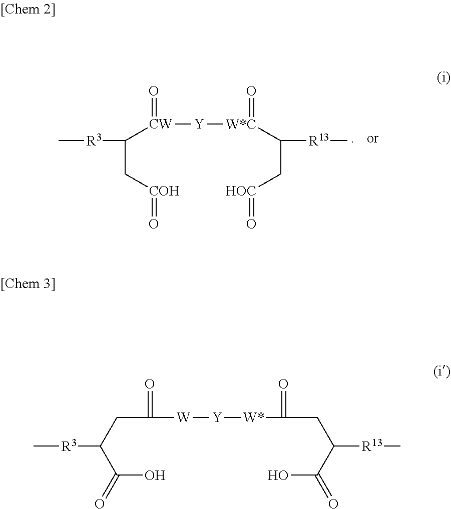

in which
W and W* are independently oxygen (O) or N—R where each R is independently hydrogen (H) or R¹
Y is a divalent group;
R¹, R¹¹, R⁴, R¹⁴, R⁵ and R15 are independently a substituted or unsubstituted hydrocarbyl group;
R³ and R¹³ are independently a divalent group;
w and ww are independently integers ranging from 0 to 1000;
x and xx are independently integers ranging from 1 to 100;
y and yy are independently integers ranging from 0 to 1000;
preferably, w and y are not simultaneously equal to 0 and ww and yy are not simultaneously equal to 0.

By “substituted” is meant that one or more hydrogen atoms of the hydrocarbyl group is (are) replaced by an atom other than hydrogen (i.e. halogen) or one or more carbon atoms is (are) replaced by an atom other than carbon such as a heteroatom like oxygen, sulfur or nitrogen.

In formula (I), the lower and upper portions are the same or different siloxanes. According to particular embodiments, each siloxane chain includes siloxane (Si—O—Si) bonds on their respective backbone. Each siloxane chain may include siloxane linkages separated by one or more divalent groups, for example a —CH₂— linking group.

Other examples of divalent groups may include polyether groups: for example, —CH₂CH₂O— linking groups (i.e., ethylene oxide group), —CH(CH₃)CH₂O— (i.e., propylene oxide), etc. Combinations of different divalent groups may be present on their respective backbones. Each of the divalent groups may be unique or repeated ie: 2 times, 5 times, 10 times, or even >10 times, etc. According to some embodiments, the siloxanes do not include a polyether group.

In various embodiments, one or both siloxanes may include at least one [SiR¹R²—O—] unit (“D” units or R*₂SiO_{2/2}). Typically, each siloxane has a repeat of D units, which typically constitute the linear portions of the siloxanes. The siloxanes also typically have terminal R*₃SiO_{1/2} units (“M” units).

In some particular embodiments, one or both of the siloxanes may optionally be branched, partially branched, and/or include a resin portion having a three-dimensional structure network. In such cases, the corresponding siloxane(s) may additionally include R*SiO_{3/2} units (“T” units) and/or SiO_{4/2} units (“Q” units). The branched or resinous character of the siloxane(s) can be attributed to the presence of T and/or Q units. The branched character of a siloxane may be attributed to side groups of one or more D units. According to particular embodiments, one or both of the siloxanes may be devoid of T units and/or units. The two siloxanes may be the same or different: i.e., one is linear and the other is branched, or both siloxanes are linear.

According to particular modes, the radicals R¹ independently denote an alkyl, aryl, alkenyl, alkaryl, or aralkyl radical. More particularly, the R¹ radicals denote an alkyl radical, preferably having from 1 to 20, from 1 to 15, from 1 to 10, from 1 to 6, from 1 to 4, or 1 or 2 carbon atoms such as, for example, methyl, ethyl, propyl, butyl, pentyl.

More particularly, all R¹ radicals denote methyl (i.e., —CH₃).

Preferably, the radical R³ is a hydrocarbylene, heterohydrocarbylene, or organoheterylene group. In particular, R³ is a (CH₂)ₙ group where n is an integer ranging from 1 to 30, 1 to 25, 1 to 20, 1 to 15, 1 to 10, 1 to 5, or 1 to 3, and more particularly equal to 3.

According to various particular modes, each of R⁴ and R⁵ may independently denote a substituted or unsubstituted hydrocarbyl group, particularly selected from those previously defined for R¹.

According to some modes, each R⁴ radical independently designates an alkyl, aryl (i.e. phenyl) or (R⁶O)ₘ group. When R⁴ is (R⁶O)ₘ, R⁶ is preferably an alkyl or aryl (ie: phenyl) radical and m is an integer from 1 to 50, 1 to 25, 1 to 10, 1 to 5, or 1. The (R⁶O)ₘ group may be a polyether group (ie: with ethylene oxide and/or propylene oxide units). According to particular modes, a radical R⁴ may be a divalent group of a silicon side chain of the siloxane.

According to a particular mode, R⁴ may denote an anhydride group of formula (1)

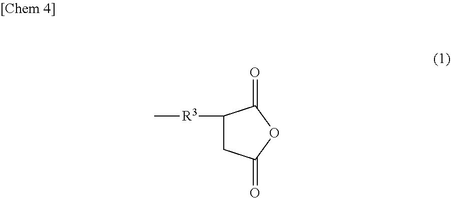

wherein R³ is as defined above.

Preferably, the R⁴ radicals are an alkyl radical or a polyether group.

Preferably, each R⁵ radical is an R1 radical. For example, each R5 and R1 is an alkyl group: i.e. methyl.

Preferably, R¹¹ is identical to or different from R¹, R¹⁴ is identical to or different from R⁴, R¹⁵ is identical to or different from R⁵. Preferably, the R⁵ radicals denote R¹ and/or the R¹⁵ radicals denote R¹¹.

Preferably, w is an integer ranging from 0 to 1000, 0 to 950, 0 to 750, 0 to 500, 0 to 400, 1 to 350, 1 to 300, 25 to 250, 50 to 200, 50 to 150, 75 to 125, 90 to 110, 90 to 100, 90 to 95, and most preferably is 93.

Preferably, x is an integer of 1 to 100, 1 to 75, 1 to 50, 1 to 25, 1 to 20, 1 to 10, or 1 to 5, and more particularly equal to 3.

Preferably, y is an integer ranging from 0 to 1000, 0 to 950, 0 to 750, 0 to 500, 0 to 400, 1 to 350, 1 to 300, 1 to 250, 1 to 200, 1 to 150, 1 to 100, 1 to 75, 1 to 50, 1 to 25, 1 to 20, 1 to 15, 1 to 10, or 1 to 5.

According to various embodiments, w and y are not simultaneously equal to 0. In some modes, the sum w+x+y ranges from 25 to 1500, 25 to 1000, 25 to 900, 25 to 800, 25 to 700, 25 to 600, 25 to 500, 25 to 400, 25 to 300, 50 to 200, 75 to 150, 85 to 125 or 90 to 110.

In some modes, x is at least 1, at least 10, at least 25, at least 50, at least 75 or at least 5.

According to particular modes, ww may be the same as or different from w, xx may be the same as or different from x, and yy may be the same as or different from y. The units of formula (I), including those represented in square brackets indexed by w, ww, x, xx, y or yy may be present in any order, randomly or in sequential form.

According to particular modes, the radicals R¹¹, which may be identical or different, denote a linear, branched or cyclic C1-C20, C1-C15, C1-C10, C1-C6, C1-C4 or C1-C2 alkyl radical such as methyl, ethyl, propyl, butyl, pentyl, etc. More preferably, the radicals R¹¹ are a methyl group.

According to particular modes, the radicals R¹⁴, which may be identical or different, denote an alkyl, aryl (i.e. phenyl) or (R¹⁶O)ₘₘ radical. When R¹⁴ is (R¹⁶O)ₘₘ, R¹⁶ is alkyl or aryl (i.e., phenyl) and ^{mm} is an integer from 1 to 50, 1 to 25, 1 to 10, 1 to 5, or 1. The group

(R¹⁶ O)ₘₘ may be a polyether group (ie: with ethylene oxide and/or propylene oxide units).

According to particular modes, the R¹⁴ radicals independently denote an alkyl radical having from 2 to 20, 2 to 15, 2 to 10, 2 to 5, or 2 carbon atoms.

According to other particular modes, R¹⁴ may be a divalent linking group of a silicon side chain of the siloxane.

According to a particular embodiment, R14 can be an anhydride group of formula (1)

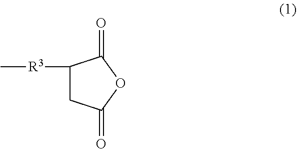

wherein R³ has the same definition as above.

Preferably, the R¹⁴ radicals denote an alkyl radical or a polyether group.

Preferably, ww is an integer ranging from 0 to 1000, 0 to 950, 0 to 750, 0 to 500, 0 to 400, 1 to 350, 1 to 300, 25 to 250, 50 to 200, 50 to 150, 75 to 125, 90 to 110, 90 to 100, 90 to 95, and most preferably 93.

Preferably, xx is an integer ranging from 1 to 100, 1 to 75, 1 to 50, 1 to 25, 1 to 20, 1 to 10, or 1 to 5, and more preferably 3.

Preferably, yy is an integer ranging from 0 to 1000, 0 to 950, 0 to 750, 0 to 500, 0 to 400, 1 to 350, 1 to 300, 1 to 250, 1 to 200, 1 to 150, 1 to 100, 1 to 75, 1 to 50, 1 to 25, 1 to 20, 1 to 15, 1 to 10 or 1 to 5.

In various embodiments, ww and yy are not simultaneously equal to 0.

According to particular embodiments, the sum ww+xx+yy ranges from 25 to 1500, 25 to 1000, 25 to 900, 25 to 800, 25 to 700, 25 to 600, 2 to 500, 25 to 400, 25 to 300, 50 to 200, 75 to 150, 85 to 125, or 90 to 110.

According to particular modes, xx is at least 1, at least 10, at least 25, at least 50, at least 75, or at least 85.

The medium X portion of formula (I) meets the following formula (i) or formula (i′):

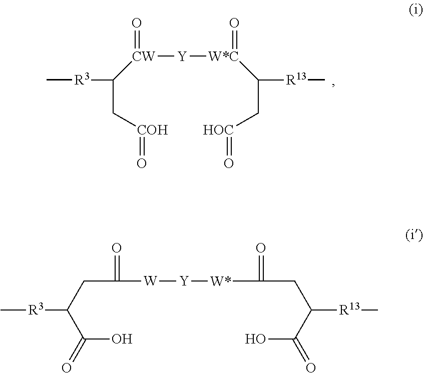

wherein
each group W and W* independently denotes an oxygen atom (O) or a group N—R, with R denoting a hydrogen atom (H) or a radical R¹; preferably R is H;
R¹³ is a divalent group, preferably is a hydrocarbylene, a heterohydrocarbylene or an organoheterylene; in particular, R¹³ designates a radical (CH₂)ₙₙ where nn is an integer from 1 to 30, from 1 to 1, from 1 to 20, from 1 to 15, from 1 to 10, from 1 to 5, from 1 to 3, and more particularly 3; R¹³ being able to be identical to or different from R³
each radical Y is a divalent group, in particular denotes a hydrocarbylene, heterohydrocarbylene or organoheterylene radical; in particular, Y is a hydrocarbylene group having from 1 to 50, from 1 to 40, from 1 to 20, from 1 to 10, from 1 to 5, from 1 to 2 carbon atoms or may denote a group of the following formula (ii)

—[Z]_{d}—[SiR⁸R¹⁰—O—]ₐ[SiR⁸R⁹—O—]_{b}[SiR⁸R¹⁰—O—]_{c}Si—[Z]_{d} (ii).

wherein
each R⁸, R⁹ independently denote a substituted or unsubstituted hydrocarbyl group, in particular selected from an alkyl group (e.g. methyl), an aryl group (e.g. phenyl), a polyether group (e.g. with ethylene oxide and propylene oxide units);
each R¹⁰ radical independently denotes a substituted or unsubstituted hydrocarbyl group such as the radicals defined above for R¹ and R⁴, in particular, R¹⁰ may be an alkyl group having from 1 to 20 carbon atoms or a polyether group or a divalent linking group of a siloxane side chain;
each Z independently comprises at least one hydrocarbylene, heterohydrocarbylene or organoheterylene group, in particular is a hydrocarbylene group having 1 to 20, 1 to 10, 1 to 5, or 1 or more carbon atoms, including Z may include siloxane linkages separated by one or more divalent groups,
e.g., a —CH₂— linkage group; other examples of divalent groups may include polyether groups: e.g., —CH₂CH₂O— (i.e, ethylene oxide group), —CH(CH₃)CH₂O— (i.e., propylene oxide group);
a is an integer ranging from 0 to 1000, 0 to 950, 0 to 750, 0 to 500, 0 to 400, 0 to 300, 0 to 200, 0 to 100, 0 to 75, 0 to 50, 0 to 25, 0 to 20, or—b is an integer ranging from 1 to 1000, 1 to 950, 1 to 750, 1 to 500, 1 to 400, 1 to 300, or 1 to 200
c is an integer ranging from 0 to 1000, from 0 to 950, from 0 to 750, from 0 to 500, from 0 to 400, from 0 to 300, from 0 to 200, from 0 to 100.
each d is equal to 0 or 1. in particular, at least one of the d's is equal to 1 or both are equal to 1; the units of formula (ii) represented in square brackets indexed by a, b, or c being present in any order, randomly or in sequential form.

According to a particular embodiment, the siloxane of formula (ii) may be a resin of formula R*ₛSiO_{(4s)/2}. Preferably, a silicone resin has along its chain T and/or Q units with M units and optionally D units. The R* radicals may be independently selected from a substituted (i.e., hydroxyl, amine functions) or unsubstituted hydrocarbonyl group and s ranges from 0 to 3. The R* groups that can be used can be those described above for the radicals R8, R9, and R10. Various combinations of such groups may be present. According to these particular cases, the silicone resin comprises a combination of M, D, T and/or Q units. In particular, it may be an MDT type resin, an MT type resin, an MDQ type resin, an MQ type resin, or an MDTQ type resin. Each of the M, D and T units may contain different R* groups. The resin may have various molecular weights such as a number average molecular weight ranging from 800 to 500,000.

According to a particular embodiment of the invention, the carboxylic acid functional silicone elastomers have a carboxyl equivalent ranging from 100 to 50,000, 500 to 10,000, or 500 to 5,000,

The carboxylic-functional silicone elastomers of formula (I) can be obtained according to one of the synthesis methods described in WO2015066161 (US20160200876).

According to a first variant of the synthesis process, certain carboxylic acid-functional silicone elastomers can be obtained by reacting 1) a first siloxane bearing pendant anhydride groups, 2) a second siloxane bearing pendant anhydride groups and 3) an organic polyol. According to a particular embodiment, one or more siloxanes different from the first two can be reacted with the above 3 reactants.

According to a second variant of the synthesis process, certain carboxylic acid-functional silicone elastomers can be obtained by reacting 1) a first siloxane bearing pendant anhydride groups, 2) a second siloxane bearing pendant anhydride groups and 3) an organic polyamine. According to a particular embodiment, one or more additional siloxanes different from the first two can be reacted with the above 3 reactants.

According to a third alternative synthesis method, certain carboxylic acid functional silicone elastomers may be obtained by reacting 1) a first siloxane bearing pendant anhydride groups, 2) a second siloxane bearing pendant anhydride groups and 3) a third siloxane having at least two hydroxyl groups. In a particular embodiment, one can make reacting with the above 3 reactants one or more additional siloxanes different from the first two.

According to a fourth alternative synthetic method, certain carboxylic acid-functional silicone elastomers may be obtained by reacting 1) a first siloxane bearing pendant anhydride groups, 2) a second siloxane bearing pendant anhydride groups, and 3) a third siloxane having pendant amine groups and/or terminal amine groups. According to a particular embodiment, one or more additional siloxanes different from the first two can be reacted with the above 3 reactants.

According to a fifth alternative synthetic method, certain carboxylic acid functional silicone elastomers may be obtained by reacting 1) a first siloxane bearing pendant hydroxyl groups, 2) a second siloxane bearing pendant hydroxyl groups, and 3) a third siloxane having at least two terminal anhydride groups. According to a particular embodiment, one or more additional siloxanes different from the first two can be reacted with the above 3 reactants.

According to a sixth alternative synthetic method, certain carboxylic acid functional silicone elastomers may be obtained by reacting 1) a first siloxane bearing pendant amine groups, 2) a second siloxane bearing pendant amine groups, and 3) a third siloxane having at least two terminal anhydride groups. According to a particular embodiment, one or more additional siloxanes different from the first two can be reacted with the above 3 reactants.

According to a seventh variant of the synthesis process, certain carboxylic acid-functional silicone elastomers can be obtained by reacting 1) a first organic alcohol, 2) a second organic alcohol and 3) a siloxane having terminal anhydride groups. According to a particular mode, one or more additional organic alcohols different from the first two can be reacted with the above 3 reactants.

Among the carboxylic acid-functional silicone elastomers of formula (I), the following compounds 1 to 12 as described respectively in Examples 4, 5, 7 to 16 of application WO2015066161 may be mentioned:

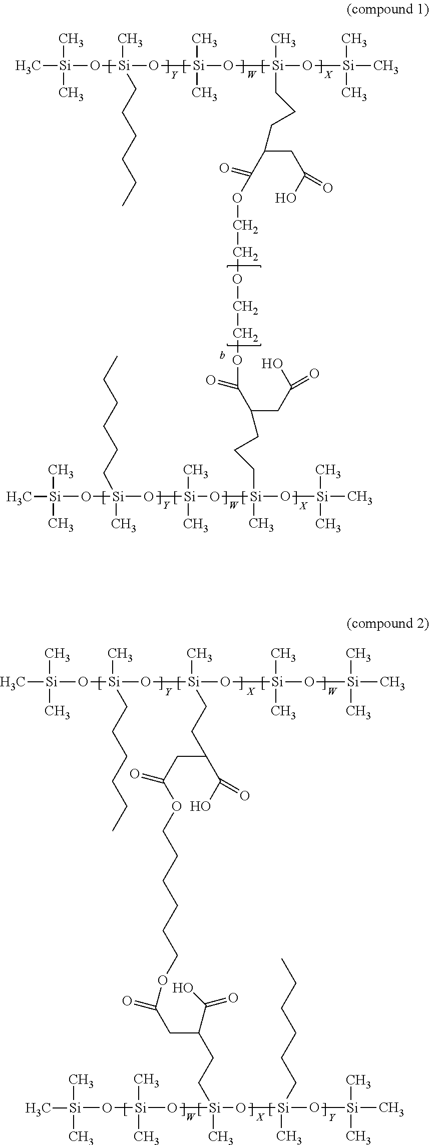

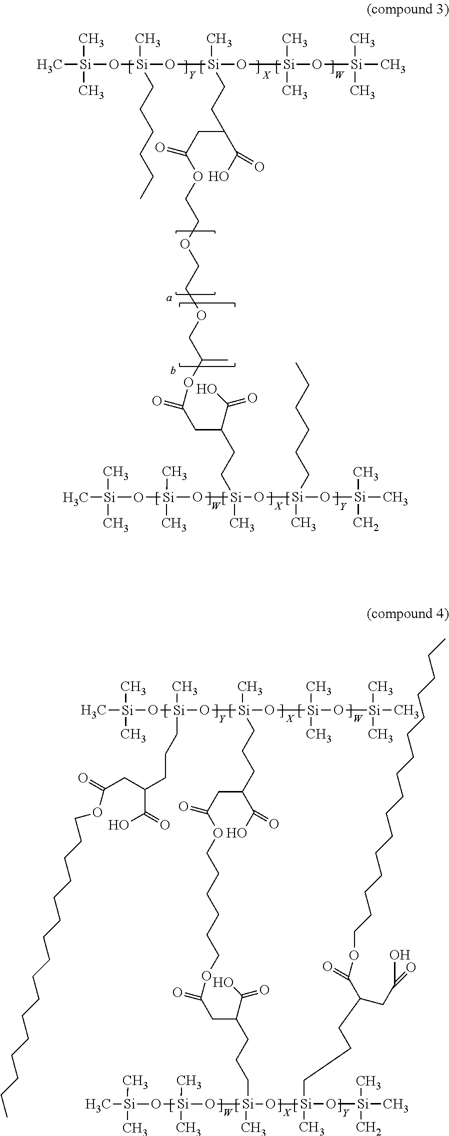

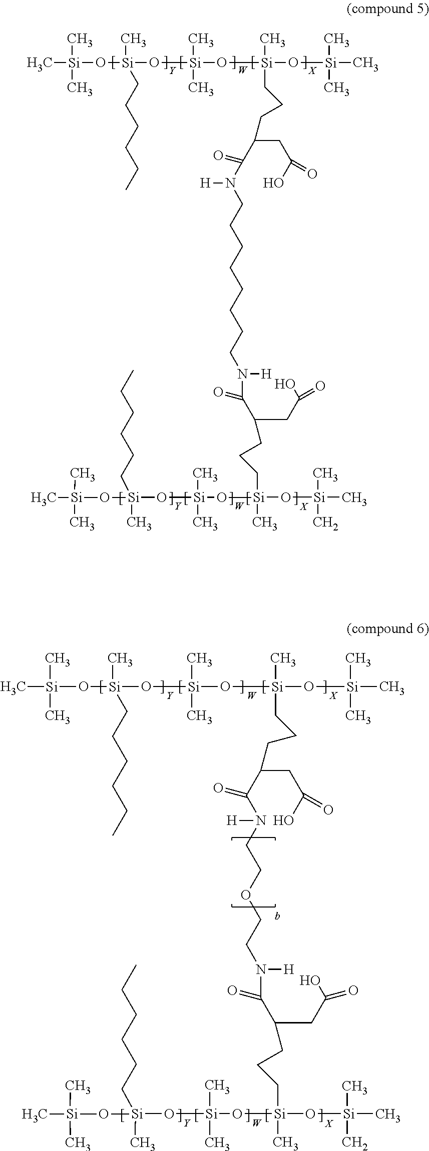

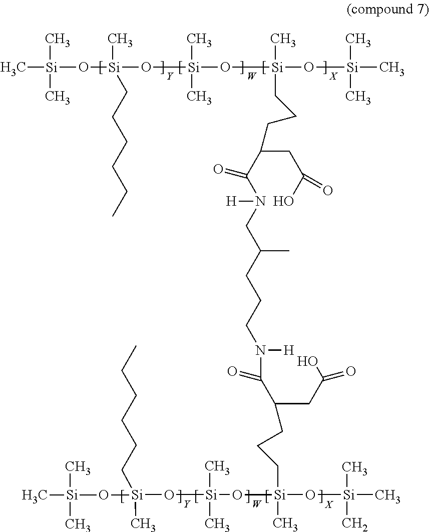

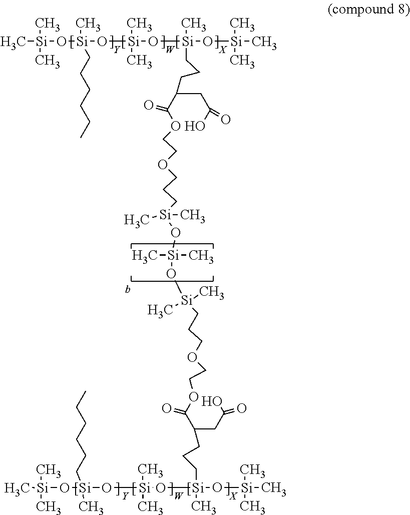

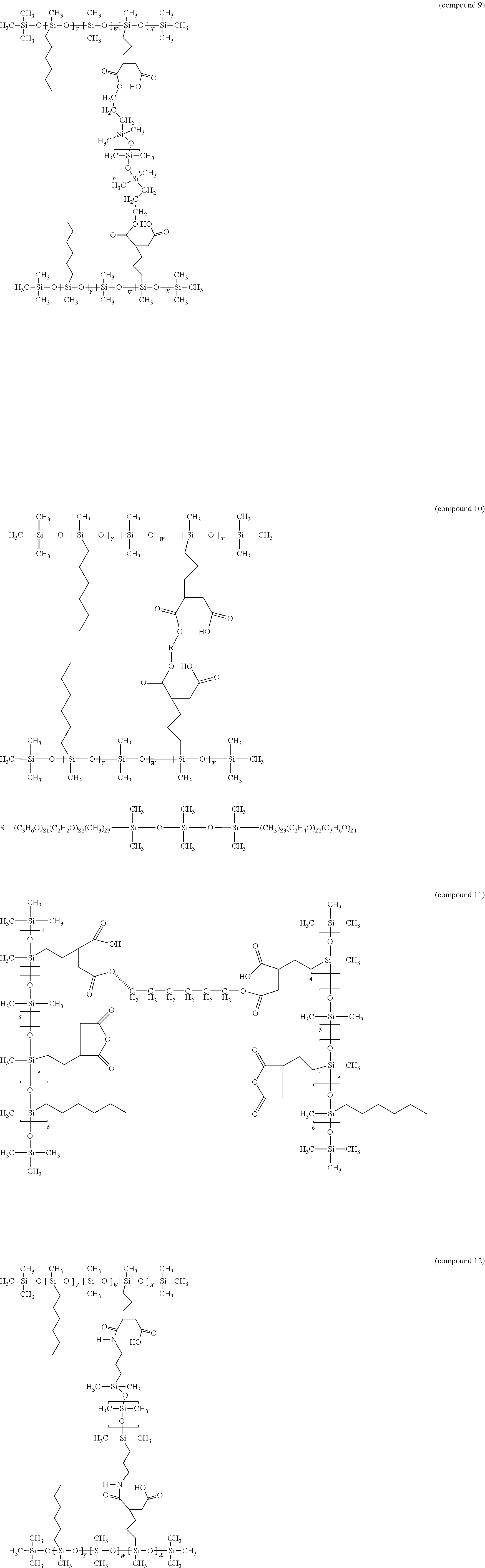

According to a particular embodiment, the composition of the invention contains at least either compound 10 or compound 11 as defined above. In particular, compound 10 is described in US20160200876 (compound of Example 15) and the examples of U.S. Pat. No. 10,918,587B2.

Among the carboxylic acid-functional silicone elastomers that can be used in the compositions of the invention, mention may also be made of the silicone elastomer with the INCI name: HEXYL/SUCCINYL DIMETHICONE CROSSPOLYMER.

According to a particular form, the carboxylic acid-functional silicone elastomers are in solid form. In particular, they may be selected from compounds 2, and 3 as defined above.

According to another particular form, the carboxylic acid-functional silicone elastomers are in dispersion in at least one H₂ oil and are in gel form.

The H₂ oil may be chosen from volatile oils and/or non-volatile oils.

By “non-volatile oil” is meant an oil remaining on the keratinous material at room temperature (25° C.) and atmospheric pressure (760 mm Hg) for at least several hours and having in particular a vapour pressure lower than 10⁻³ mm Hg (0.13 Pa).

The oil or oils H₂ may be selected from hydrocarbon oils, silicone oils and mixtures thereof.

For the purposes of the present invention, “silicone oil” means an oil comprising at least one Si—O group, and more particularly an organopolysiloxane.

Among the volatile H₂ silicone oils which can be used in the compositions, mention may be made, for example, of volatile linear or cyclic silicone oils, in particular those having a viscosity of less than or equal to 8 centistokes (8 10⁻⁶ mm²/s), and in particular having from 2 to 7 silicon atoms, these silicones optionally comprising alkyl or alkoxy groups having from 1 to 10 carbon atoms.

As a linear H₂ volatile silicone oil suitable for use in the invention, mention may be made of octamethyltrisiloxane, decamethyltetrasiloxane, dodecamethylpentasiloxane, and mixtures thereof.

As a cyclic volatile H₂ silicone oil suitable for use in the invention, cyclomethicones such as octamethylcyclotetrasiloxane, decamethylcyclopentasiloxane, dodecamethylcyclohexasiloxane, and mixtures thereof may be mentioned.

Examples of H₂ linear non-volatile silicone oils include polydimethylsiloxanes (also known as dimethicones); alkyl dimethicones; vinylmethylmethicones and polydimethylsiloxanes modified with aliphatic groups and/or functional groups such as hydroxyl, thiol, carboxylic acid and/or amine groups.

As examples of H₂ linear non-volatile hydrocarbon oils, mention may be made of oils selected from the non-volatile hydrocarbon solvents with alcohol, carboxylic acid, amine, or amide functions as mentioned above and recommended in the oil phase of the composition.

According to a preferred form of the invention, the H₂ oil or oils may be selected from volatile hydrocarbon oils as described previously for the H₁ oils. The H2 oil or oils may be the same as or different from the H₁ oil or oils present in the oil phase of the composition.

According to a particular form of the invention, the H₂ oil or oils present in the carboxylic acid-functional silicone elastomer oil dispersion is/are identical to the H₁ oil or oils present in the oil phase of the composition.

According to a more particularly preferred form of the invention, the oil H₂ present in the oily dispersion of carboxylic acid-functional silicone elastomer and the oil H₁ present in the oily phase of the composition denote a C8-C16 isoalkane of petroleum origin, and more particularly isododecane.

According to a preferred form, the carboxylic acid-functional silicone elastomer oil dispersion comprises from 10 to 40% by weight of silicone elastomer active material, and more preferably from 20 to 35% by weight relative to the total weight of the oil dispersion.

According to a preferred embodiment, the composition of the invention comprises at least one carboxylic acid-functional silicone elastomer dispersed in isododecane and in gel form. In particular, said silicone elastomer is selected from compounds 1, 4 to 11 as defined above.

According to a particularly preferred form, the composition of the invention comprises compound 10 or compound 11 in dispersion in isododecane and in gel form, and more particularly in an active material content equal to 30% by weight with respect to the total weight of the dispersion.

According to another particularly preferred form, the composition of the invention comprises a carboxylic acid-functional silicone elastomer in dispersion in isododecane and in gel form with the INCI name: ISODODECANE (and) HEXYL/SUCCINYL DIMETHICONE CROSSPOLYMER and more particularly in an active material content equal to 30% by weight relative to the total weight of the dispersion such as the commercial product DOWSIL EL-7314 SILICONE ELASTOMER BLEND® sold by the company Dow Corning comprising 30% by weight of active material of said silicone elastomer.

According to a preferred embodiment of the invention, the ratio by weight of the amount of carboxylic acid functional silicone elastomer to the total amount of volatile hydrocarbon solvent(s) ii) ranges from 1.5 to 5.

Silicone Resins and/or Silicone Resin Copolymers

[According to a particular embodiment, the composition of the invention may additionally comprise at least one silicone resin and/or at least one dimethiconol silicone silicone resin copolymer.

### A) Silicone Resin

The nomenclature for silicone resins (also known as siloxane resins or silicone resins) is known as “MDTQ”, the resin being described in terms of the various siloxane monomer units it comprises, each of the letters “MDTQ” characterizing a type of unit.

The letter “M” represents the Monofunctional unit of formula R1R2R3SiO_{1/2}, the silicon atom being connected to a single oxygen atom in the polymer comprising this unit.

The letter “D” signifies a Difunctional unit R1R2SiO_{2/2} in which the silicon atom is bonded to two oxygen atoms

The letter “T” represents a trifunctional unit of the formula R1SiO_{3/2}.

Such resins are described, for example, in “Encyclopedia of Polymer Science and Enginnering, vol. 15, John and Wiley and Sons, New York, (1989), pp. 265-270, and U.S. Pat. Nos. 2,676,182, 3,627,851, 3,772,247, 5,248,739 or U.S. Pat. Nos. 5,082,706, 5,319,040, 5,302,685 and 4,935,484.

In the units M, D, T defined above, R, namely R1, R2 and R3, represents a hydrocarbon radical (in particular alkyl) having from 1 to 10 carbon atoms, a phenyl group, a phenylalkyl group or even a hydroxyl group.

Finally, the letter “Q” signifies a SiO_{4/2} tetrafunctional unit in which the silicon atom is bonded to four oxygen atoms which are themselves bonded to the rest of the polymer.

Various silicone resins with different properties can be obtained from these different units, the properties of these polymers varying according to the type of monomers (or units), the nature and number of the R radical, the length of the polymeric chain, the degree of branching and the size of the pendant chains.

As silicone resins which can be used in the compositions according to the invention, it is possible to use, for example, silicone resins of type MQ, of type T or of type MQT.

### MQ Resins:

As an example of MQ type silicone resins, mention may be made of alkylsiloxysilicates of the formula [(R1)₃SiO_{1/2]x}(SiO_{4/2})_{y} (MQ units) in which x and y are integers ranging from 50 to 80, and such that the R1 group represents a radical as defined above, and preferably is an alkyl group having from 1 to 8 carbon atoms, or a hydroxyl group, preferably a methyl group.

As examples of MQ silicone resins of the trimethylsiloxysilicate type, mention may be made of those marketed under the reference SR1000® by the General Electric company, under the reference TMS 803® by the Wacker company, under the name “KF-7312J®” by the Shin-Etsu company, “DC 749®”, “DC 593®” by the Dow Corning company.

As silicone resins comprising MQ siloxysilicate units, mention may also be made of phenylalkyl siloxysilicate resins, such as phenylpropyldimethylsiloxysilicate (Silshine 151® marketed by the General Electric company). The preparation of such resins is described in particular in U.S. Pat. No. 5,817,302.

### T Resins:

Examples of T-type silicone resins include polysilsesquioxanes of the formula (RSiO_{3/2})ₓ (T-units) wherein x is greater than 100 and such that the R moiety is an alkyl moiety having from 1 to 10 carbon atoms, said polysilsesquioxanes may further include Si—OH end groups.

Preferably, polymethylsilsesquioxane resins in which R represents a methyl group, such as those marketed:
by the company Wacker under the reference Resin MK® such as Belsil PMS MK®: polymer comprising CH₃SiO_{3/2} repeating units (T units), which may also comprise up to 1% by weight of (CH₃)₂SiO_{2/2} units (D units) and having an average molecular weight of about 10,000 g/mol, or
by the company SHIN-ETSU under the references KR 220L® which are composed of T units of formula CH₃SiO_{3/2} and have Si OH (silanol) end groups, under the reference KR-242A® which comprise 98% of T units and 2% of dimethyl D units and have Si—OH end groups, or under the reference KR 251® comprising 88% of T units and 12% of dimethyl D units and have Si—OH end groups.

### MQT Resins:

As resins comprising MQT units, those cited in U.S. Pat. No. 5,110,890 are known in particular.

A preferred form of MQT-type resins are MQT-propyl resins (also called MQTPr). Such resins suitable for use in the compositions according to the invention include those described and prepared in WO 2005/075542, the contents of which are incorporated herein by reference.

The MQ-T-propyl resin preferably comprises the units:
(i)(R31SiO_{1/2})ₐ
(ii) (R22SiO_{2/2})_{b}
(iii) (R3SiO_{3/2})_{c} and
(iv) (SiO_{4/2})_{d}
with R1, R2 and R3 representing independently a hydrocarbon radical (in particular alkyl) having from 1 to 10 carbon atoms, a phenyl group, a phenylalkyl group or even a hydroxyl group and preferably an alkyl radical having from 1 to 8 carbon atoms or a phenyl group
a, b, c and d being molar fractions,
a being between 0.05 and 0.5,
b being between zero and 0.3,
c being greater than zero,
d being between 0.05 and 0.6,

$$a + b + c + d = 1 ,$$

provided that more than 40 mole percent of the R3 groups in the siloxane resin are propyl groups.

Preferably the siloxane resin comprises the units:
(i)(R1₃SiO_{1/2})ₐ
(ii) (R2₂SiO_{2/2})_{b}
(iii) (R3SiO_{3/2})_{c} and
(iv) (SiO_{4/2})_{d}
with R1 and R3 independently representing an alkyl group having from 1 to 8 carbon atoms, R1 preferably being a methyl group and R3 preferably being a propyl group, a being between 0.05 and 0.5, preferably between 0.15 and 0.4
c being greater than zero, preferably between 0.15 and 0.4
d being between 0.05 and 0.6, preferably between 0.2 and 0.6, or between 0.2 and 0.55
a+b+c+d=1, and a, b, c and d being mole fractions,
provided that more than 40 mole percent of the R3 groups in the siloxane resin are propyl groups.

Siloxane resins suitable for use according to the invention are obtainable by a process comprising reacting:
A) an MQ resin comprising at least 80 mol % of (R1₃SiO_{1/2})ₐ units and (SiO_{4/2})_{d} units, where R1 is an alkyl group having from 1 to 8 carbon atoms, an aryl group, a carbinol group or an amino group,
a and d being greater than zero, the ratio a/d being between 0.5 and 1.5; and
B) a propyl resin T comprising at least 80 mole percent of (R3SiO_{3/2})_{c} units where R3 is an alkyl group having from 1 to 8 carbon atoms, an aryl group, a carbinol group or an amino group,
c being greater than zero,
with the proviso that at least 40 mole percent of the R3 groups are propyl groups, wherein the A/B weight ratio is between 95:5 and 15:85, preferably the A/B weight ratio is 30:70.

Advantageously, the A/B mass ratio is between 95:5 and 15:85. Preferably, the A/B ratio is less than or equal to 70:30. These preferred ratios have been shown to provide comfortable deposition.

Preferably, the composition according to the invention comprises, as silicone resin, at least one resin of the MQ type, more particularly of the Trimethylsiloxysilicate type, such as those marketed under the reference SR1000® by the General Electric company, under the reference TMS 803® by the Wacker company, under the name “KF-7312J®” by the Shin-Etsu company, “DC 749®”, “DC 593®” by the Dow Corning company.

According to a particular embodiment of the invention, the silicone resin is present in the composition in a resin solids content ranging from 8 to 40% by weight based on the total weight of the composition, preferably ranging from 10 to 35% by weight and more preferably from 15 to 30% by weight based on the weight of the composition.

### B/ Silicone Resin-Dimethiconol Type Silicone Copolymer.

Such copolymers are described, for example, in “Silicone Pressure Sensitive Adhesive”, Sobieski and Tangney, Handbook of Pressure Sensitive Adhesive Technology (D. Satas Ed.), Von Nostrand Reinhold, New York.

In the copolymer, the silicone resin is present at a level of between 45% and 75% (based on total silicone weight) and the dimethiconol silicone is present at a level of between 25% and 55%, with the sum of the percentages of silicone resin and dimethiconol silicone being equal to 100. Preferably, the silicone resin is present at a level between 55% and 65% (based on total silicone mass) and the dimethiconol type silicone is present at a level between 35% and 45%, with the sum of the percentages of silicone resin and dimethiconol type silicone equaling 100.

Preferably, the silicone resin according to the invention is the condensation product of SiO₂ groups and R₃(SiO)_{1/2} (triorganosilyl) groups wherein each R group is independently selected from methyl, ethyl, propyl or vinyl radicals and wherein the ratio of SiO₂ functions to R₃(SiO)_{1/2} functions of the silicone resin ranges from 0.6 to 0.9.

Triorganosilyl groups that can be used to form the silicone resin can be trimethylsilyl, triethylsilyl, methylmethylproprylsilyl, dimethylvinylsilyl units and mixtures thereof. The trimethylsilyl moiety is preferred in the context of the invention.

Preferably, the dimethiconol silicone according to the invention is an OH-terminated diorganopolysiloxane having a viscosity of between 100 and 100,000 mm² (cst) at 25° C. wherein the diorganopolysiloxane substituents are independently selected from methyl, ethyl, propyl or vinyl. The diorganopolysiloxanes are preferably linear polymers. Examples of diorganopolysiloxane may be, but are not limited to, a polydimethylsiloxane, an ethylmethylpolysiloxane, a copolymer of dimethylsiloxane and methylvinylsiloxane, and mixtures of such polymers or copolymers having OH ends. The preferred diorganopolysiloxane is a polydimethylsiloxane.

Examples of the synthesis of such a copolymer are, for example, described in U.S. Pat. No. 5,162,410 or in CA Patent 711756.

The copolymers according to the present invention can thus be prepared by heating the following mixture:
from 45% to 75% by weight of silicone resin being the condensation product of SiO₂ and R₃(SiO)_{1/2} units for which each R group is independently selected from methyl, ethyl, propyl or vinyl radicals and for which the ratio of SiO₂ to R₃(SiO)_{1/2} functions of the silicone resin ranges from 0.6 to 0.9;
from 25% to 55% by weight of OH-terminated diorganopolysiloxane having a viscosity of from 100 to 100,000 mm² (cst) at 25° C. wherein the substituents of the diorganopolysiloxane are independently selected from methyl, ethyl, propyl, or vinyl;
from 0.001% to 5% of a suitable catalyst, which is preferably an organic aliphatic amine compound preferably selected from primary amines, secondary amines, tertiary amines, carboxylic acid salts of the aforementioned amines and quaternary ammonium salts.

The mixture is heated to a temperature between 80° C. and 160° C. until the adhesive character of the resulting silicone copolymer is achieved.

Preferred copolymers according to the invention are those with the INCI name TRIMETHYLSILOXYSILICATE/DIMETHICONOL CROSSPOLYMER such as the products marketed by Dow Corning under the references DOWSIL FC 5002 ID RESIN GUM®, DOWSIL FC-5001 CM RESIN GUM®, DOWSIL FC 5004 RESIN GUM®, DOWSIL FC 5002 ID RESIN GUM® (Dow Corning Chemical Company); and more specifically DOWSIL FC 5002 ID RESIN GUM®/DOW CORNING

The composition according to the invention advantageously has a content of silicone resin-based copolymer and dimethiconol-type silicone ranging from 0.2 to 60% by weight based on the weight of the composition. Preferably, this content ranges from 1 to 30% by weight based on the weight of the composition.

More specifically, the silicone resin and dimethiconol type silicone copolymer(s) may in particular be present in the composition according to the invention in a content of more than 1.0% and up to 40% by weight relative to the total weight of the composition, preferably ranging from 1.5 to 20% by weight, and preferably ranging from 1.5 to 15% by weight.

### Silicone Polyamide

According to a particular embodiment, the composition of the invention may additionally comprise at least one silicone polyamide.

The silicone polyamides are preferably solid at room temperature (25° C.) and atmospheric pressure (760 mm Hg).

By polymer is meant in the sense of the invention a compound having at least 2 repeat units, preferably at least 3 repeat units and more preferably 10 repeat units.

The silicone polyamides of the composition of the invention may be polymers of the polyorganosiloxane type such as, for example, those described in U.S. Pat. Nos. 5,874,069, 5,919,441, 6,051,216 and 5,981,680.

According to the invention, the silicone polymers may belong to the following two families:
(1) polyorganosiloxanes having at least two amide groups, these two groups being located in the polymer chain, and/or
(2) polyorganosiloxanes having at least two amide groups, these two groups being located on grafts or branches.

According to a first embodiment, the silicone polymers are polyorganosiloxanes as defined above and whose units capable of establishing hydrogen interactions are arranged in the polymer chain.

More particularly, the silicone polymers may be polymers comprising at least one unit of the general formula (II):

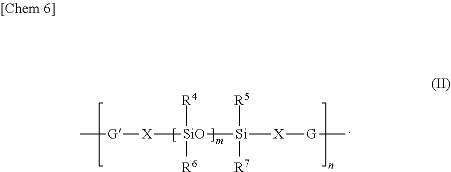

in which: G′ represents C(O) when G represents —C(O)—NH—Y—NH—, and G′ represents —NH— when G represents —NH—C(O)—Y—C(O)—
R⁴, R⁵, R⁶ and R⁷, which may be identical or different, represent a group selected from:
linear, branched or cyclic, saturated or unsaturated C1 to C40 hydrocarbon groups, which may contain in their chain one or more oxygen, sulfur and/or nitrogen atoms, and which may be partially or totally substituted by fluorine atoms
C6 to C10 aryl groups, optionally substituted by one or more C1 to C4 alkyl groups
polyorganosiloxane chains which may or may not contain one or more oxygen, sulfur and/or nitrogen atoms
the X's, which may be identical or different, represent a linear or branched C1 to C30 alkylene di-yl group which may contain one or more oxygen and/or nitrogen atoms in its chain,
Y is a divalent linear or branched, saturated or unsaturated C1-C50 alkylene, arylene, cycloalkylene, alkylarylene or arylalkylene group, which may contain one or more oxygen, sulfur and/or nitrogen atoms, and/or bear as substituent one of the following atoms or groups of atoms: fluorine, hydroxy, C3-C8 cycloalkyl, C1-C40 alkyl, C5-C10 aryl, phenyl optionally substituted with 1 to 3 C1-C3 alkyl, C1-C3 hydroxyalkyl and C1-C6 aminoalkyl groups, or
Y is a group of the formula:

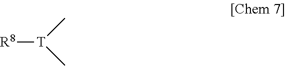

in which
T represents a trivalent or tetravalent, linear or branched, saturated or unsaturated, C3 to C24 hydrocarbon group optionally substituted by a polyorganosiloxane chain, and which may contain one or more atoms selected from O, N and S, or T represents a trivalent atom selected from N, P and Al, and
R⁸ represents a linear or branched C1-C50 alkyl group or a polyorganosiloxane chain, which may contain one or more ester, amide, urethane, thiocarbamate, urea, thiourea and/or sulfonamide groups which may or may not be bonded to another polymer chain,
n is an integer ranging from 2 to 500, preferably from 2 to 200, and m is an integer ranging from 1 to 1000, preferably from 1 to 700 and more preferably from 6 to 200.

Preferably, m is an integer ranging from 50 to 150.

According to one embodiment of the invention, 80% of the R⁴, R⁵, R⁶ and R⁷, of the polymer are preferably selected from methyl, ethyl, phenyl and 3,3,3-trifluoropropyl groups.

According to another mode, 80% of R⁴, R⁵, R⁶ and R⁷, of the polymer are methyl groups.

According to the invention, Y may represent various divalent groups, optionally additionally having one or two free valencies to establish bonds with other units of the polymer or copolymer.

Preferably, Y represents a group selected from:
C1 to C20 linear alkylene groups, preferably C1 to C10,
branched alkylene groups which may comprise rings and non-conjugated unsaturations, with C30 to C56,
C5 to C6 cycloalkylene groups
phenylene groups optionally substituted by one or more C1 to C40 alkyl groups
C1 to C20 alkylene groups with 1 to 5 amide groups,
C1-20 alkylene groups, having one or more substituents, selected from hydroxyl, C3-8 cycloalkane, C1-3 hydroxyalkyl and C1-6 alkylamines,
polyorganosiloxane chains of the formula:

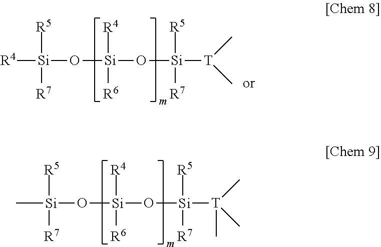

wherein R⁴, R⁵, R⁶, R⁷, T and m are as defined above.

According to the second embodiment, the polyorganosiloxanes may be polymers comprising at least one unit having the formula (III):

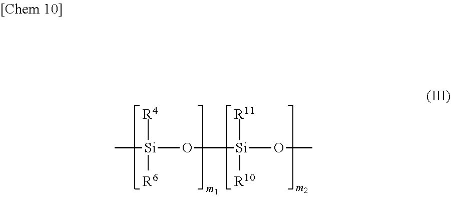

wherein R⁴ and R⁶, the same or different, are as defined above for formula (II), R¹⁰ represents a group as defined above for R⁴ and R⁶, or represents the group of formula X G R¹² in which X and G are as defined above for formula (II) and R¹² represents a hydrogen atom or a linear, branched or cyclic, saturated or unsaturated, C1 to C50 hydrocarbon group optionally containing in its chain one or more atoms selected from O, S and N, optionally substituted by one or more fluorine atoms and/or one or more hydroxyl groups, or a phenyl group optionally substituted by one or more C1 to C4 alkyl groups
R¹¹ represents the group of formula X G R₁₂ wherein X, G and R¹² are as defined above,
m₁ is an integer from 1 to 998, and
m₂ is an integer ranging from 2 to 500.

According to the invention, the silicone polymer used as a structuring agent may be a homopolymer, i.e. a polymer comprising a plurality of identical units, in particular units of formula (II) or of formula (III).

According to the invention, a silicone polymer consisting of a copolymer having a plurality of different units of the formula (II) can also be used, i.e. a polymer in which at least one of R⁴, R⁵, R⁶, R⁷, X, G, Y, m and n is different in one of the units.

Alternatively, the copolymer may be formed from a plurality of units of formula (II), wherein at least one of R⁴, R⁶, R¹⁰, R¹¹, m₁ and m₂ is different in at least one of the units.

Alternatively, a polymer comprising at least one unit of formula (II) and at least one unit of formula (III) may be used, wherein the units of formula (II) and the units of formula (III) may be the same as or different from each other.

According to an alternative embodiment of the invention, a polymer may also be used comprising in addition at least one hydrocarbon unit having two groups capable of establishing hydrogen interactions selected from ester, amide, sulfonamide, carbamate, thiocarbamate, urea, urethane, thiourea, oxamido, guanidino, biguanidino groups and combinations thereof.

These copolymers can be block polymers, block polymers or graft polymers.

According to an advantageous embodiment of the invention, the groups capable of establishing hydrogen interactions are amide groups of formula —C(O)NH and —HN C(O). In this case, the structuring agent may be a polymer comprising at least one unit of formula (IV) or (V):

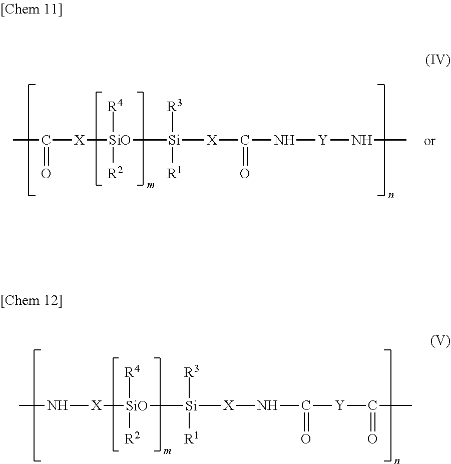

wherein R⁴, R⁵, R⁶, R⁷, X, Y, m and n are as defined above.

In these polyamides of formula (IV) or (V), m ranges from 1 to 700, in particular from 15 to 500 and in particular from 50 to 200 and n ranges in particular from 1 to 500, preferably from 1 to 100 and most preferably from 4 to 25; and
X is preferably a linear or branched alkylene chain having from 1 to 30 carbon atoms, in particular from 1 to 20 carbon atoms, in particular from 5 to 15 carbon atoms and more particularly from 10 carbon atoms, and
Y is preferably a linear or branched alkylene chain or one that may have rings and/or unsaturation, having from 1 to 40 carbon atoms, in particular from 1 to 20 carbon atoms, and more preferably from 2 to 6 carbon atoms, in particular from 6 carbon atoms.

In formulas (IV) and (V), the alkylene group representing X or Y may optionally contain in its alkylene portion at least one of the following:
1 to 5 amide, urea, urethane, or carbamate groups,
a C5 or C6 cycloalkyl group, and
a phenylene group optionally substituted with 1 to 3 identical or different C1 to C3 alkyl groups.

In formulas (IV) and (V), the alkylene groups may also be substituted with at least one member selected from the group consisting of:
a hydroxy group,
one C3 to C8 cycloalkyl group
one to three C1 to C40 alkyl groups,
a phenyl group optionally substituted with one to three C1 to C3 alkyl groups,
a C1 to C3 hydroxyalkyl group, and
a C1-C6 aminoalkyl group.

In these formulae (IV) and (V), Y may also represent:

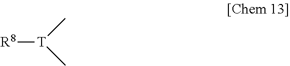

wherein R⁸ represents a polyorganosiloxane chain, and T represents a group of formula:

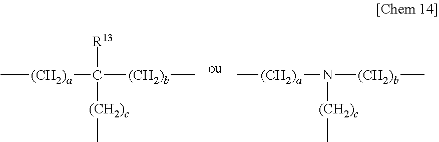

wherein a, b and c are independently integers ranging from 1 to 10, and R¹³ is hydrogen or a group as defined for R⁴, R⁵, R⁶ and R⁷.

In formulae (IV) and (V), R⁴, R⁵, R⁶ and R⁷ are preferably, independently, a linear or branched C1-C40 alkyl group, preferably a CH₃, C₂H₅, n-C₃H₇ or isopropyl group, a polyorganosiloxane chain or a phenyl group optionally substituted with one to three methyl or ethyl groups.

According to a preferred embodiment, the silicone polyamide comprises at least one unit of formula (IV) and/or (V).

As discussed above, the polymer may comprise the same or different units of formula (IV) or (V).

Thus, the polymer may be a polyamide containing a plurality of units of formula (IV) or (V) of different lengths, or a polyamide having the following formula (VI):

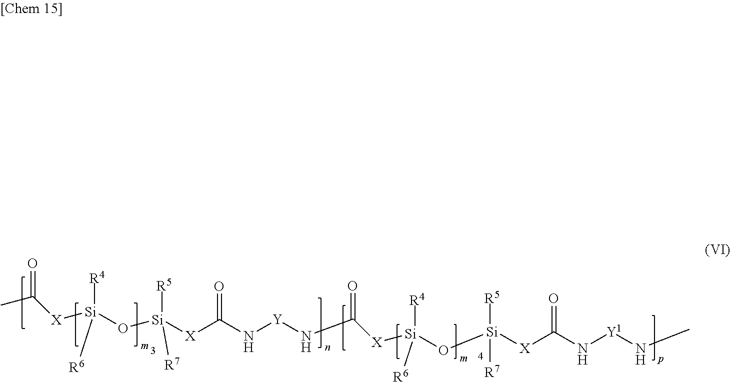

wherein X, Y, n, R⁴ to R⁷ have the meanings given above, m₁ and m₂, which are different, are selected from the range of 1 to 1000, and p is an integer from 2 to 300.

In this formula, the units may be structured to form either a block copolymer, a random copolymer, or an alternating copolymer. In this copolymer, the units may not only be of different lengths but also of different chemical structures, for example having different Y's. In this case, the polymer may have the formula (VII):

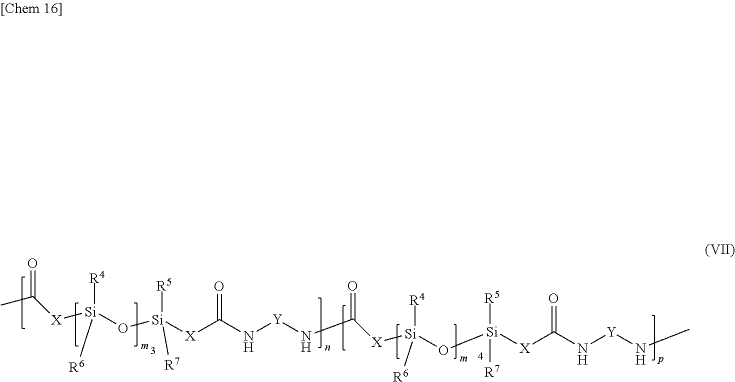

wherein R⁴ to R⁷, X, Y, m₁, m₂, n and p have the meanings given above and Y1 is different from Y but selected from the groups defined for Y.

As before, the individual units may be structured to form either a block copolymer, a random copolymer, or an alternating copolymer.

In this first embodiment of the invention, the structuring agent may also consist of a graft copolymer. Thus, the polyamide with silicone units can be grafted and optionally crosslinked with silicone chains with amide groups. Such polymers can be synthesized with trifunctional amines.

In this case, the polymer may comprise at least one unit of the following formula (VIII):

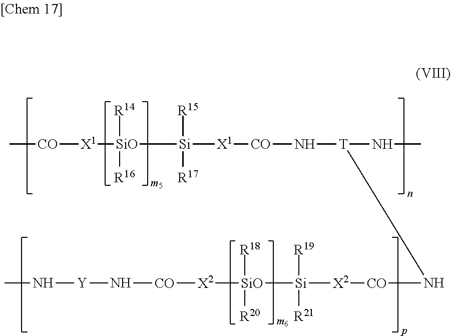

wherein X¹ and X², which are the same or different, have the meaning given for X in formula (II), n is as defined in formula (II), Y and T are as defined in formula (III), R¹⁴ to R²¹ are groups selected from the same group as R⁴ to R⁷, m₁ and m₂ are numbers in the range of 1 to 1,000, and p is an integer from 2 to 500.

In formula (VIII), it is preferred that:
p is from 1 to 25, more preferably from 1 to 7,
R¹⁴ to R²¹ are methyl groups,
T has one of the following formulae:

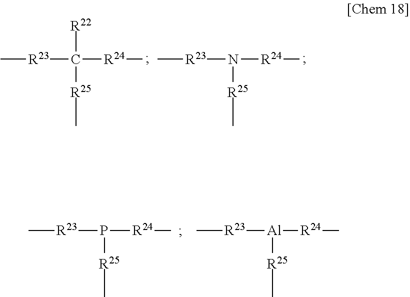

wherein R²² is hydrogen or a group selected from the groups defined for R⁴ to R⁷, and R²³, R²⁴ and R²⁵ are independently linear or branched alkylene groups, preferably further to the formula:

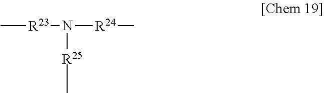

in particular with R²³, R²⁴ and R²⁵ being CH₂CH₂,
m₁ and m₂ are from 15 to 500, more preferably from 15 to 45,
X¹ and X² represent (CH₂)₁₀, and
Y is CH₂.

These grafted silicone unit polyamides of formula (VIII) can be copolymerized with silicone polyamides of formula (III) to form block copolymers, alternating copolymers or random copolymers. The weight percentage of grafted silicone units (VIII) in the copolymer can range from 0.5 to 30 weight percent.

According to the invention, as discussed above, the siloxane units may be in the main or backbone chain of the polymer, but they may also be present in grafted or pendant chains. In the main chain, the siloxane units may be in the form of segments as described above. In the pendant or grafted chains, the siloxane units may appear individually or in segments.

According to an alternative embodiment of the invention, a copolymer of silicone polyamide and hydrocarbon polyamide may be used, that is, a copolymer comprising units of formula (III) or (IV) and hydrocarbon polyamide units. In this case, the silicone polyamide units may be arranged at the ends of the hydrocarbon polyamide.

According to a preferred embodiment, the silicone polyamide comprises units of formula IV:

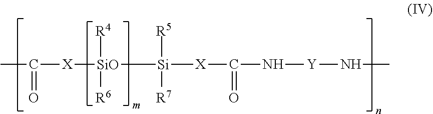

wherein R⁴, R⁵, R⁶ and R⁷ independently represent a linear or branched C1-C40 alkyl group, preferably a CH₃, C₂H₅, n-C₃H₇ or isopropyl group, a polyorganosiloxane chain or a phenyl group optionally substituted with one to three methyl or ethyl groups, and m ranges from 1 to 700, in particular from 15 to 500 and in particular from 50 to 200, and n ranges in particular from 1 to 500, preferably from 1 to 100 and most preferably from 4 to 25.

Preferably, according to this embodiment, the groups R⁴, R⁵, R⁶ and R⁷ represent methyl groups, one of X and Y represents an alkylene group of 6 carbon atoms and the other an alkylene group of 11 carbon atoms, n representing the degree of polymerization (DP) of the polymer.

As examples of such silicone polyamides, mention may be made of the compounds marketed by the Dow Corning company under the name DC 2-8179 GELLANT® (DP 100) and DC 2-8178 GELLANT® (DP 15), the INCI name of which is “NYLON-611/DIMETHICONE COPOLYMER”.

Advantageously, the composition according to the invention comprises at least one polydimethylsiloxane block polymer of the general formula (II) having an index m value of about 100.

The index “m” corresponds to the degree of polymerization of the silicone portion of the polymer.

Even more preferably, the composition according to the invention comprises at least one polymer comprising at least one unit of formula (III) wherein m ranges from 50 to 200, in particular from 75 to 150, and preferably of the order of 100.

Still preferably R⁴, R⁵, R⁶ and R⁷ independently represent a linear or branched C1-C40 alkyl group, preferably a CH₃, C₂H₅, n-C₃H₇ or isopropyl group in formula (IV).

As an example of a usable silicone polymer, one of the silicone polyamides, obtained according to Examples 1 to 3 of U.S. Pat. No. 5,981,680, may be mentioned.

According to a preferred mode, a polyamide silicone polymer with the INCI name: NYLON-611/DIMETHICONE COPOLYMER marketed by the Dow Corning company under the name DC 2-8179 GELLANT® (DP 100) is used.

According to an embodiment of the invention, the polymer consists of a homopolymer or copolymer comprising urethane or urea groups. These polymers are described in detail in application WO 2003/106614 published on 24 Dec. 2003, the contents of which are incorporated in the present application by reference.

As before, such a polymer may comprise polyorganosiloxane units containing two or more urethane and/or urea groups, either in the polymer backbone or on side chains or as pendant groups.

The polymers and/or copolymers used in the composition of the invention advantageously have a transition temperature from the solid state to the liquid state ranging from 45 to 190° C. Preferably, they have a transition temperature from the solid state to the liquid state ranging from 70 to 130° C. and better from 80 to 105° C.

The content of silicone polyamide, expressed as active material, preferably varies from 8 to 30% by weight, more preferably from 10 to 25% by weight, more particularly from 12 to 25% by weight (expressed as active material), based on the weight of the composition.

### Pulverulent Colorants

According to a particular form of the invention, the composition according to the invention additionally comprises at least one pulverulent colorant.

The pulverulent colorants may be chosen from mineral pigments, organic pigments, pearlescent materials and mixtures thereof.

By “pigments” is meant white or colored, mineral or organic particles, insoluble in an aqueous medium, intended to color and/or opacify the resulting composition and/or deposit. These pigments may be white or colored, mineral and/or organic.

According to a particular embodiment, the pigments used according to the invention are chosen from mineral pigments.

By “mineral pigment” is meant any pigment which meets the definition of the Ullmann encyclopedia in the chapter on inorganic pigment. Examples of mineral pigments useful in the present invention include zirconium or cerium oxides, as well as zinc, iron (black, yellow or red) or chromium oxides, manganese violet, ultramarine blue, chromium hydrate and ferric blue, titanium dioxide, metal powders such as aluminum powder and copper powder. The following inorganic pigments can also be used: Ta₂O₅, Ti₃O₅, Ti₂O₃, TiO, ZrO₂ in mixture with TiO₂, ZrO₂, Nb₂O₅, CeO₂, ZnS.

The size of the pigment useful in the present invention is generally greater than 100 nm and can be up to 10 μm, preferably from 200 nm to 5 μm, and more preferably from 300 nm to 1 μm.

According to a particular embodiment of the invention, the pigments have a size characterized by a D[50] greater than 100 nm and may be up to 10 μm, preferably from 200 nm to 5 μm, and more preferably from 300 nm to 1 μm.

The sizes are measured by static light scattering using a commercial granulometer of the Master Sizer 3000® type from Malvern, allowing the granulometric distribution of all the particles to be apprehended over a wide range that can go from 0.01 to 1000 μm. The data are processed on the basis of the classical Mie diffusion theory. This theory is most suitable for size distributions ranging from submicron to multimicron, it allows to determine an “effective” particle diameter. This theory is described in Van de Hulst, H. C., “Light Scattering by Small Particles”, Chapters 9 and 10, Wiley, New York, 1957.

D[50] represents the maximum size that 50% by volume of the particles presents.

According to a particular embodiment of the invention, the inorganic pigment comprises a lipophilic or hydrophobic coating.

According to a particular embodiment of the invention, the pigments may be coated according to the invention with at least one compound selected from metal soaps; N-acylated amino acids or their salts; lecithin and its derivatives; isopropyl trisostearyl titanate; isostearyl sebacate; natural vegetable or animal waxes; polar synthetic waxes; fatty esters; phospholipids; silicone surfactants such as organopolysiloxanes, alkylalkoxysilanes such as triethoxycaprylylsilane; fluorinated surfactants such as perfluoroalkyl phosphates, perfluoropolyethers, polytetrafluoropolyethylenes (PTFE), perfluoroalkanes, perfluoroalkyl silazanes, hexafluoropropylene polyoxides, polyorganosiloxanes comprising perfluoroalkyl perfluoropolyether groups; fluorosilicone surfactants such as perfluoroalkyl dimethicones, perfluoroalkyl silanes and perfluoroalkyl trialkoxysilanes; and mixtures thereof.

According to a preferred mode, the pigments may be coated according to the invention with an N-acylated amino acid or a salt thereof which may comprise an acyl group having from 8 to 22 carbon atoms, such as a 2-ethyl hexanoyl, caproyl, lauroyl, myristoyl, palmitoyl, stearoyl, cocoyl group.

The amino acid may be, for example, lysine, glutamic acid or alanine. The salts of these compounds may be aluminum, magnesium, calcium, zirconium, zinc, sodium, potassium salts. Thus, according to a particularly preferred embodiment, the pigments may be coated with an N-acylated amino acid derivative may be in particular a glutamic acid derivative and/or a salt thereof, and more particularly a stearoyl glutamate, such as aluminum stearoyl glutamate. As examples of pigments treated with aluminum stearoyl glutamate, mention may be made of the titanium dioxide pigments and the black, red and yellow iron oxide pigments sold under the trade name NAI® by the company by MIYOSHI KASEI.

According to a preferred mode, the pigments may be coated according to the invention with isopropyl titanium triisostearate. As examples of pigments treated with isopropyl titanium triisostearate (ITT), mention may be made of those sold under the trade reference BWBO-I2® (Iron Oxide C177499 and Isopropyl Titanium Triisostearate), BWYO-I2® (Iron Oxide C177492 and Isopropyl Titanium Triisostearate) and BWRO-I2® (Iron Oxide C177491 and Isopropyl Titanium Triisostearate) by the KOBO company.

The pigments usable according to the invention may also be organic pigments.

By “organic pigment” is meant any pigment which meets the definition of the Ullmann encyclopedia in the chapter on organic pigment. The organic pigment may in particular be selected from nitroso, nitro, azo, xanthene, quinoline, anthraquinone, phthalocyanine, metal complex compounds, isoindolinone, isoindoline, quinacridone, perinone, perylene, diketopyrrolopyrrole, thioindigo, dioxazine, triphenylmethane, quinophthalone.

The organic pigment(s) may be chosen, for example, from carmine, carbon black, aniline black, melanin, azo yellow, quinacridone, phthalocyanine blue, sorghum red, the blue pigments codified in the Color Index under the references C1 42090, 69800, 69825, 73000, 74100, 74160, the yellow pigments coded in the Color Index under the references C 11680, 11710, 15985, 19140, 20040, 21100, 21108, 47000, 47005, the green pigments coded in the Color Index under the references C1 61565 61570, 74260, the orange pigments coded in the Color Index as C111725, 15510, 45370, 71105, the red pigments coded in the Color Index as C 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indolic, phenolic derivatives as described in patent FR2 679 771.

These pigments may also be in the form of composite pigments such as described in patent EP1184426. These composite pigments may be composed in particular of particles comprising an inorganic core covered at least partially with an organic pigment and at least one binder ensuring the attachment of the organic pigments to the core.

The pigment may also be a lacquer. By lacquer is meant insolubilized dyes adsorbed on insoluble particles, the assembly thus obtained remaining insoluble during use.

The inorganic substrates on which the dyes are adsorbed are, for example, alumina, silica, calcium sodium borosilicate or calcium aluminum borosilicate, and aluminum.

Among the organic dyes, cochineal carmine may be mentioned. Mention may also be made of the products known by the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090).

As examples of lacquers, mention may be made of the product known under the name D&C Red 7 (CI 15 850:1).

Preferably, the composition according to the invention comprises at least one pulverulent coloring material of the mineral pigment type, in particular chosen from metal oxides, and more particularly chosen from titanium dioxides, iron oxides, whether coated or not, and mixtures thereof.

The pearlescent pigments can be chosen from white pearlescent pigments such as mica coated with titanium or bismuth oxychloride, colored pearlescent pigments such as titanium mica with iron oxides, titanium mica with, in particular, ferric blue or chromium oxide, titanium mica with an organic pigment of the aforementioned type, as well as pearlescent pigments based on bismuth oxychloride.

Preferably, the pulverulent colorant(s) is/are present in the composition in a content ranging from 5 to 40% by weight, preferably from 10% to 30% by weight, more particularly from 15 to 25% by weight relative to the total weight of the composition.

### Aqueous Phase

According to a particular embodiment, the composition further comprises an aqueous phase.

The aqueous phase comprises water and optionally water soluble or miscible ingredients such as water soluble solvents.

A water suitable for the invention may be a floral water such as cornflower water and/or a mineral water such as VITTEL water, LUCAS water or LA ROCHE POSAY water and/or a thermal water.

By “water-soluble solvent” is meant in the present invention a compound which is liquid at room temperature and miscible with water (miscibility in water greater than 50% by weight at 25° C. and atmospheric pressure).

The water-soluble solvents usable in the composition of the invention may furthermore be volatile.

Among the water-soluble solvents which can be used in the composition according to the invention, mention may be made in particular of ethanol, glycols having from 2 to 8 carbon atoms such as ethylene glycol, propylene glycol, 1,3-butylene glycol, propanediol, pentylene glycol, glycerine and dipropylene glycol, C3-C4 ketones and C2-C4 aldehydes.

According to a particular embodiment, the water is present in a concentration ranging from 5 to 90 wt %, and more particularly from 10 to 80 wt %, based on the total weight of said composition.

According to a particular embodiment, the aqueous phase of the composition is present in a concentration ranging from 5 to 90 wt %, and more particularly from 10 to 80 wt %, based on the total weight of said composition.

According to a particular embodiment of the invention, the ratio by weight of the total amount of volatile hydrocarbon oil(s) relative to the total weight of the composition to the amount of aqueous phase relative to the total weight of the composition is greater than 0.085.

According to a particular form, the non-solid compositions of the invention comprising an aqueous phase can be in various galenic forms such as
emulsions, in particular oil-water or water-oil emulsions in which one of the two oily and aqueous phases is dispersed in the other so-called continuous phase in the form of droplets and the composition is macroscopically homogeneous to the naked eye;
macroscopically homogeneous bi-gels to the naked eye comprising a gelled aqueous phase and a gelled oil phase as described in patent EP29858541 B1.

### Additives

The compositions according to the invention may comprise, in addition to the additives commonly used in care and/or make-up products, such as lipophilic active ingredients, hydrophilic active ingredients in the case of a composition comprising an aqueous phase, such as vitamins, solar UV filters, moisturizing agents such as polyols such as glycerol, propanediol, pentylene glycol, liposoluble coloring matters; lipophilic thickening or gelling agents; hydrophilic thickening or gelling agents in the case of a composition comprising an aqueous phase; preservatives, perfumes and their mixtures.

### Oil-Soluble Colorants

A composition according to the invention may additionally comprise at least one oil-soluble colorant and preferably in an amount of at least 0.01% by weight relative to the total weight of the composition.

By “oil-soluble colorant”, within the meaning of the invention, is meant any generally organic, natural or synthetic compound which is soluble in an oily phase or in solvents miscible with a fatty substance and is capable of coloring.

For obvious reasons, this quantity is likely to vary significantly with respect to the intensity of the desired color effect and the color intensity provided by the coloring materials considered, and its adjustment is clearly within the competence of the person skilled in the art.

As oil-soluble dyes suitable for the invention, mention may be made in particular of synthetic or natural fat-soluble dyes such as, for example, DC Red 17, DC Red 21, DC Red 27, DC Green 6, DC Yellow 11, DC Violet 2, DC Orange 5, Sudan red, carotenes (-carotene, lycopene), xanthophylls (capsanthin, capsorubin, lutein), palm oil, Sudan brown, quinoline yellow, rocou, curcumin.

### Water-Soluble Colorants

A composition according to the invention may additionally comprise at least one water-soluble colorant and preferably in an amount of at least 0.01% by weight based on the total weight of the composition

By “water-soluble colorant”, within the meaning of the invention, is meant any generally organic, natural or synthetic compound which is soluble in an aqueous phase or in water-miscible solvents and is capable of coloring.

As water-soluble dyes suitable for the invention, mention may be made in particular of synthetic or natural water-soluble dyes such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beet), carmine, copper chlorophyllin, methylene blue, anthocyanins (enocianine, black carrot, hibiscus, elderberry), caramel, riboflavin.

Examples of water-soluble colorants are beet juice and caramel.

### Cosmetic Compositions

The present invention also relates to a cosmetic composition comprising, in a physiologically acceptable medium, a composition as defined above.

By “physiologically acceptable medium” is meant a medium particularly suitable for the application of a composition of the invention to the skin.

The physiologically acceptable medium is generally adapted to the nature of the support to which the composition is to be applied, as well as to the appearance in which the composition is to be packaged.

According to a particularly preferred form, the composition of the invention is anhydrous.

By “anhydrous”, within the meaning of the present invention, is meant a composition comprising a content of less than or equal to 2% by weight of water, preferably less than or equal to 1%, more preferably less than 0.5% by weight relative to the total weight of the said composition, or even free of water. If necessary, such small quantities of water may in particular be provided by the ingredients of the composition which may contain residual quantities thereof.

The non-solid compositions may also have an aqueous phase and be in a galenic form in which the oil phase and the aqueous phase form a macroscopically homogeneous mixture, i.e. the two phases are not observable to the naked eye.

Such a galenic form may be an oil-in-water or water-in-oil emulsion or a bi-gel such as those described in patent EP29858541 B1.

The non-solid compositions of the invention may be in the form of milks, creams or gels.

### Applications

According to one embodiment, a composition of the invention may advantageously be in the form of a skin, body or facial care composition, in particular for the face.

According to another embodiment, a composition of the invention may advantageously be in the form of a make-up composition for the keratinous materials, in particular the skin of the body or of the face, in particular the face.

Thus, according to a sub-mode of this embodiment, a composition of the invention can advantageously be in the form of a base composition for makeup.

A composition of the invention may advantageously be in the form of a foundation.

Such compositions are in particular prepared according to the general knowledge of the man of the art.

Throughout the description, including the claims, the expression “comprising a” should be understood as being synonymous with “comprising at least”.

The invention is further illustrated by the examples and figures presented below. Unless otherwise indicated, the amounts shown are expressed as percent by weight.

### Examples 1, 11 to 18, 23 and 24 (Out of Invention) and Examples 2 to 10, 19 to 22 (Invention)

The following compositions were prepared.

TABLE 1

| | Ex1 | | | | | | | | | |
|---|---|---|---|---|---|---|---|---|---|---|
| | out of | Ex2 | Ex3 | Ex4 | Ex5 | Ex6 | Ex7 | Ex8 | Ex9 | Ex10 |
| Ingredients | invention | invention | invention | invention | invention | invention | invention | invention | invention | invention |
| ISODODECANE (and) | 47.6 | 47.6 | 47.6 | 47.6 | 47.6 | 47.6 | 47.6 | 47.6 | 47.6 | 47.6 |
| HEXYL/SUCCINYL | | | | | | | | | | |
| DIMETHICONE | | | | | | | | | | |
| CROSSPOLYMER | | | | | | | | | | |
| (DOWSIL EL-7314 | | | | | | | | | | |
| SILICONE | | | | | | | | | | |
| ELASTOMER | | | | | | | | | | |
| BLEND ®/DOW | | | | | | | | | | |
| CORNING. | | | | | | | | | | |
| under gel form in a | | | | | | | | | | |
| dispersion at 30% by | | | | | | | | | | |
| weight in isododecane) | | | | | | | | | | |
| OCTYLDODECANOL | | 4.8 | | | | | | | | |
| 2-HEXYLDECANOL | | | 4.8 | | | | | | | |
| C12-C13 ALKYL | | | | 4.8 | | | | | | |
| LACTATE | | | | | | | | | | |
| OLEIC ACID | | | | | 4.8 | | | | | |
| LINOLEIC ACID | | | | | | 4.8 | | | | |
| DILINOLEIC ACID | | | | | | | 4.8 | | | |
| DIOCTYLAMINE | | | | | | | | 4.8 | | |
| TRIOCTYLAMINE | | | | | | | | | 4.8 | |
| ISOPROPYL | | | | | | | | | | 4.8 |
| LAUROYL | | | | | | | | | | |
| SARCOSINATE | | | | | | | | | | |
| ISODODECANE | qsp 100 | qsp 100 | qsp 100 | qsp 100 | qsp 100 | qsp 100 | qsp 100 | qsp 100 | qsp 100 | qsp 100 |
| Weight ratio of | 16.9. | 16.9. | 16.9. | 16.9. | 16.9. | 16.9. | 16.9. | 16.9. | 16.9. | 16.9. |
| the total amount of | | | | | | | | | | |
| volatile oil(s) to | | | | | | | | | | |
| the total amount | | | | | | | | | | |
| of hydrocarbonated | | | | | | | | | | |
| liquid solvent | | | | | | | | | | |

TABLE 2

| | Ex11 | Ex12 | Ex13 | Ex14 | Ex15 | Ex16 | Ex17 | Ex18 |
|---|---|---|---|---|---|---|---|---|
| | out of | out of | out of | out of | out of | out of | out of | out of |
| Ingredients | invention | invention | invention | invention | invention | invention | invention | invention |
| ISODODECANE (and) | 47.6 | 47.6 | 47.6 | 47.6 | 47.6 | 47.6 | 47.6 | 47.6 |
| HEXYL/SUCCINYL | | | | | | | | |
| DIMETHICONE | | | | | | | | |
| CROSSPOLYMER | | | | | | | | |
| (DOWSIL EL-7314 | | | | | | | | |
| SILICONE | | | | | | | | |
| ELASTOMER | | | | | | | | |
| BLEND ®/DOW | | | | | | | | |
| CORNING. | | | | | | | | |
| under gel form in a | | | | | | | | |
| dispersion at 30% by | | | | | | | | |
| weight in isododecane | | | | | | | | |
| HUILE DE RICIN | 4.8 | | | | | | | |
| Molecular | | | | | | | | |
| weight >600 g/mol | | | | | | | | |
| DICAPRYLYL ETHER | | 4.8 | | | | | | |
| DIMETHYL | | | 4.8 | | | | | |
| ISOSORBIDE (ETHER) | | | | | | | | |
| DICAPRYLYL | | | | 4.8 | | | | |
| CARBONATE | | | | | | | | |
| CAPRYLIC/CAPRIC | | | | | 4.8 | | | |
| TRIGLYCERIDE | | | | | | | | |
| (ESTER) | | | | | | | | |
| ISOPROPYL | | | | | | 4.8 | | |
| MYRISTATE | | | | | | | | |
| (ESTER) | | | | | | | | |
| ACETYL TRIBUTYL | | | | | | | 4.8 | |
| CITRATE | | | | | | | | |
| (ESTER) | | | | | | | | |
| DIISOPROPYL | | | | | | | | 4.8 |
| ADIPATE | | | | | | | | |
| (ESTER) | | | | | | | | |
| ISODODECANE | qsp 100 | qsp 100 | qsp 100 | qsp 100 | qsp 100 | qsp 100 | qsp 100 | qsp 100 |
| Weight ratio of the | 16.9. | 16.9. | 16.9. | 16.9. | 16.9. | 16.9. | 16.9. | 16.9. |
| total amount of | | | | | | | | |
| volatile oil(s) to | | | | | | | | |
| the total amount of | | | | | | | | |
| hydrocarbonated | | | | | | | | |
| liquid solvent | | | | | | | | |

TABLE 3

| | Ex19 | Ex20 | Ex21 | Ex22 |
|---|---|---|---|---|
| Ingrédients | invention | invention | invention | invention |
| ISODODECANE (and) | 47.6 | 47.6 | 47.6 | 47.6 |
| HEXYL/SUCCINYL | | | | |
| DIMETHICONE | | | | |
| CROSSPOLYMER | | | | |
| (DOWSIL EL-7314 | | | | |
| SILICONE | | | | |
| ELASTOMER | | | | |
| BLEND ®/ | | | | |
| DOW CORNING. | | | | |
| under gel form in a | | | | |
| dispersion at 30% by | | | | |
| weight in isododecane | | | | |
| ETHANOL | 4.8 | | | |
| ISOPROPYL | | 4.8 | | |
| ALCOHOL | | | | |
| HEXANOL | | | 4.8 | |
| NONANOL | | | | 4.8 |
| ISODODECANE | qsp 100 | qsp 100 | qsp 100 | qsp 100 |
| Weight ratio of the | 16.9. | 16.9. | 16.9. | 16.9. |
| total amount of volatile | | | | |
| oil(s) to the total amount | | | | |
| of hydrocarbonated | | | | |
| liquid solvent | | | | |

TABLE 4

| | Ex 23 | Ex 24 |
|---|---|---|
| | out of | out of |
| Ingrédients | invention | invention |
| ISODODECANE (and) HEXYL/ | 47.6% | 47.6% |
| SUCCINYL DIMETHICONE | | |
| CROSSPOLYMER | | |
| (DOWSIL EL-7314 SILICONE | | |
| ELASTOMER BLEND ®/ | | |
| DOW CORNING. | | |
| under gel form in a dispersion | | |
| at 30% by weight in isododecane | | |
| ETHANOL | 3.6 | |
| ISOPROPYL ALCOHOL | | 3.6 |
| ISODODECANE | 48.8 | 48.8 |
| Weight ratio of the total amount | 22.8 | 22.8 |
| of volatile oil(s) to the total | | |
| amount of hydrocarbonated | | |
| liquid solvent | | |

### Operating Mode

Formulations 1 to 24 were carried out with a Triblab® or Olsa Minilab® type reactor from the company VMI. An ice bath was placed under the beaker to keep the manufacturing temperature below 2000.

The silicone elastomer was pre-dispersed under shear. The rest of the ingredients were then added to the elastomer little by little until a homogeneous composition was obtained. The formula was then debubbled under a vacuum bell jar.

### Rheological Analysis Under Shear Stress

Rheological measurements were performed on each of Examples 1-10 in an imposed stress rheometer (RheoStress 600®, Haake). Shear stress and velocity gradient measurements are performed using a cone sensor (C35/2° Ti L; 222-1871; 35 mm diameter, 2° angle), with a 0.105 mm air gap and a measurement plane (MPC 35; 222-1549; 35 mm diameter, sandblasted).

The air gap temperature was controlled using an Eheim Pro 3600/2075020 hydraulic circulation pump and a Haake Universal Temperature Controller System (UTC) 6001.

An anti-evaporation bell was positioned above the cone plane and the plane during the measurement.

The temperature of the air gap was set at 25±0.5° C. A 120 second isotherm at this temperature is applied before each measurement.

An upward oscillation curve with an amplitude sweep was imposed with a stress ramp from 10-3 to 103 Pa, with a frequency set at 1 Hz with 50 steps.

### Discussion and Results

The rheological behavior of formulas 1 through 24 was analyzed. Measurements of storage modulus G′, loss or dissipation modulus G″ were used to determine the complex modulus G* as well as the phase shift δ between stress and strain. These data were analyzed as a function of shear stress.

The behavior of the rheological curves showed that these are materials that only flowed if a sufficiently high stress was applied to them.

The value of the complex modulus G* at the plateau before the curve breaks gives us an idea of the consistency of the product at rest.

The rheological data of the formulae 1 to 24 are described in the tables below:

TABLE 5

| Rheological | Ex1 | | | | | | | | | |
|---|---|---|---|---|---|---|---|---|---|---|
| measurements | out of | Ex2 | Ex3 | Ex4 | Ex5 | Ex6 | Ex7 | Ex8 | Ex9 | Ex10 |
| and aspect | invention | invention | invention | invention | invention | invention | invention | invention | invention | invention |
| hardness | Non | 0.15 | 0.19 | 0.08 | 0.19 | 0.10 | 0.10 | 0.18 | 0.29 | 0.03 |
| (in Newtons) | measurable | | | | | | | | | |
| G* at the | Non | 275 | 500 | 100 | 180 | 195 | 120 | 100 | 310 | 75 |
| plateau | measurable | | | | | | | | | |
| (in Pa) | | | | | | | | | | |
| Aspect | The | Uniform | Uniform | Uniform | Uniform | Uniform | Uniform | Uniform s | Uniform | Uniform gel |
| | composition | consistent | consistent | consistent | consistent | consistent | consistent | consistent | consistent | that flows |
| | shifted in | gel | gel | gel | gel | gel | gel | gel | gel | but of |
| | phase | | | | | | | | | sufficient |
| | | | | | | | | | | consistency |
| | | | | | | | | | | to allow |
| | | | | | | | | | | good |
| | | | | | | | | | | sampling |
| | | | | | | | | | | and |
| | | | | | | | | | | spreading |

TABLE 6

| Rheological | Ex11 | Ex12 | Ex13 | Ex14 | Ex15 | Ex16 | Ex17 | Ex18 |
|---|---|---|---|---|---|---|---|---|
| measurements | out of | out of | out of | out of | out of | out of | out of | out of |
| and aspect | invention | invention | invention | invention | invention | invention | invention | invention |
| hardness | Non applicable | Non applicable | Non applicable | Non applicable | Non applicable | Non applicable | Non applicable | Non applicable |
| (in Newtons) | heterogenous | heterogenous | heterogenous | heterogenous | heterogenous | heterogenous | heterogenous | heterogenous |
| G* at the | Non | Non | Non | Non | Non | Non | Non | Non |
| plateau | measurable | measurable | measurable | measurable | measurable | measurable | measurable | measurable |
| (in Pa) | | | | | | | | |
| Aspect | The | The | The | The | The | The | The | The |
| | composition | composition | composition | composition | composition | composition | composition | composition |
| | shifted in | shifted in | shifted in | shifted in | shifted in | shifted in | shifted in | shifted in |
| | phase | phase | phase | phase | phase | phase | phase | phase |

TABLE 7

| Rheological | | | | |
|---|---|---|---|---|
| measurements | Ex19 | Ex20 | Ex21 | Ex22 |
| and aspect | invention | invention | invention | invention |
| hardness | 0.04 | 0.09 | 0.26 | 0.10 |
| (in Newtons) | | | | |
| G* at | 70 | 550 | 500 | 720 |
| the plateau | | | | |
| (in Pa) | | | | |
| Aspect | Uniform gel | Uniform gel | Uniform gel | Uniform gel |
| | that flows | that allow | that allow | that allow |
| | but of | good | good | good |
| | sufficient | sampling | sampling | sampling |
| | consistency | and | and | and |
| | to allow | spreading | spreading | spreading |
| | good | | | |
| | sampling and | | | |
| | spreading | | | |

TABLE 8

| Rheological | | |
|---|---|---|
| measurements | Ex23 | Ex24 |
| and aspect | out of invention | out of invention |
| hardness | 0.03 | 0.05 |
| (in Newtons) | | |
| G* at the plateau | 20 | 35 |
| (in Pa) | | |
| Aspect | Uniform gel that | Uniform gel that |
| | flows but of weak | flows but of weak |
| | consistency which | consistency which |
| | does not allow | does not allow |
| | good sampling and | good sampling and |
| | spreading | spreading |

The results showed that Examples 2 to 10 and 19 to 22 of the invention comprising hydrocarbon solvents with an alcohol, carboxylic acid, amine or amide function and with a molecular mass of less than 600 g/mol had a sufficiently flexible, homogeneous consistency to be taken up and easily spread on the keratinous material to be covered, in particular the skin, contrary to example 1 outside the invention not containing this type of solvent and to examples 11 to 18 outside the invention comprising other types of hydrocarbon solvents having a molecular mass greater than 600 g/mol (castor oil), or comprising an ether, carbonate or ester function.

As observed in Examples 20-22 according to the invention comprising a primary alcohol comprising at least 3 carbon atoms, the consistency was increased compared to Example 19 comprising a primary alcohol comprising 2 carbon atoms.

The results also showed that Examples 23 and 24 outside the invention in which the ratio by weight of the total amount of volatile oil(s) to the total amount of hydrocarbon liquid solvent(s) ii) was strictly greater than 20.0 had a consistency that was too weak to be picked up and easily spread on the keratinous material to be coated, especially the skin

1. Non-solid composition for caring for and/or making up keratinous materials, in particular the skin, comprising, preferably in a physiologically acceptable medium:
a) an oily phase comprising
i) at least one apolar volatile hydrocarbon oil H₁ and
ii) at least one liquid hydrocarbon solvent with a molecular mass of less than 600 g/mol comprising at least one alcohol, carboxylic acid, amine or amide function and mixtures thereof; and
b) at least one silicone elastomer with carboxylic acid functions;
the ratio by weight of the total amount of volatile oil(s) to the total amount of liquid hydrocarbon solvent(s) ii) being less than or equal to 20.0.

2. Composition according to claim, wherein the apolar volatile hydrocarbon oil H₁ is selected from C8-C16 isoalkanes of petroleum origin, and more particularly isododecane

3. Composition according to claim 1 or 2, wherein the apolar volatile hydrocarbon oil H₁ is selected from volatile linear alkanes comprising from 6 to 14 carbon atoms.

4. Composition according to claim 4, wherein the apolar volatile hydrocarbon oil H₁ is selected from:
a mixture of n-dodecane and n-tetradecane;
a mixture of volatile linear C9-C12 alkanes of INCI name: C9-12 ALKANE; or
a mixture of n-undecane (C11) and n-tridecane C13).

5. Composition according to any one of the preceding claims, wherein the apolar volatile hydrocarbon oil(s) H₁ is (are) present in contents ranging from 50 to 90 wt %, and more preferably ranging from 70 to 90 wt % based on the total weight of the oil phase.

6. Composition according to any one of the preceding claims, wherein the apolar volatile hydrocarbon oil(s) H₁ is (are) present in an amount ranging from 45 to 85% by weight, and more preferably from 55 to 75% by weight based on the total weight of the composition.

7. Composition according to any one of the preceding claims, wherein the liquid hydrocarbon solvent(s) comprising at least one alcohol, carboxylic acid, amine and/or amide function has (have) a molecular weight of less than 500 g/mol, more preferably less than 400 g/mol, and more particularly less than 300 g/mol.

8. Composition according to any one of the preceding claims, wherein the liquid hydrocarbon solvent is ethanol.

9. Composition according to any one of the preceding claims, wherein the liquid hydrocarbon solvent or solvents is/are selected from primary alcohols comprising at least 3 carbon atoms, with a molecular weight of less than 500 g/mol, preferably 400 g/mol, and more preferably less than 300 g/mol, and in particular selected from isopropanol, octyldodecanol, n-hexanol, 1-nonanol, 2-hexyldecanol, oleic alcohol, and mixtures thereof.

10. Composition according to any one of claims 1 to 8, wherein the liquid hydrocarbon solvent or solvents is/are selected from secondary alcohols of the lactic acid ester and C12-C15 monohydric alcohol type, in particular selected from lauryl lactate, myristyl lactate, C12-C13 alkyl lactate, C12-C15 alkyl lactate, and more particularly C12-C13 alkyl lactate.

11. Composition according to any one of claims 1 to 8, wherein the liquid hydrocarbon solvent or solvents is/are selected from unsaturated fatty acids with a molecular weight of less than 600 g/mol, in particular selected from oleic acid, linoleic acid, dilinoeic acid, linolenic acid, and mixtures thereof

12. Composition according to any one of claims 1 to 8, wherein the liquid hydrocarbon solvent or solvents is/are selected from secondary dialkylamines and tertiary trialkyamines with a molecular weight of less than 600 g/mol, in particular selected from dioctylamine, trioctylamine, and mixtures thereof.

13. Composition according to any one of claims 1 to 8, wherein the liquid hydrocarbon solvent or solvents comprising at least one amid, function is/are selected from Isopropyl Lauroyl Sarcosinate, N-acetyl N-butylaminopropionate, N,N Dimethyldecanamide, N,N Dimethylcaprylamide and mixtures thereof, and more particularly Isopropyl Lauroyl Sarcosinate.

14. Composition of any one of the preceding claims, wherein the ratio by weight of the total amount of volatile oil(s) based on the total weight of the composition to the total amount of liquid solvent(s) ii) based on the total weight of the composition] ranges from 5 to 20.

15. Composition according to any one of the preceding claims, wherein the liquid hydrocarbon solvent(s) with alcohol, carboxylic acid, amine or amide function is (are) present in contents ranging from 1 to 20% by weight, and more preferably ranging from 2 to 15% by weight based on the total weight of the composition.

16. Composition according to any one of the preceding claims, comprising a carboxylic acid-functional silicone elastomer active material content ranging from 2 to 25 wt %, preferably ranging from 5 to 20 wt % based on the weight of the composition.

17. Composition according to any one of the preceding claims, where the silicone elastomer with carboxylic acid functions is chosen from those corresponding to the following formula (1): 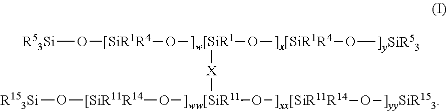
in which the radical X has the following formula (i) or formula (i′): 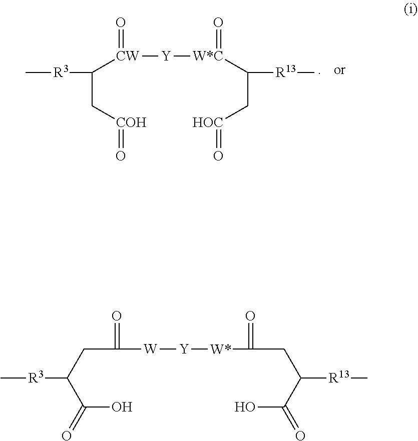
in which
W and W* independently denote an oxygen atom (O) or a group N—R where each radical R independently denotes a hydrogen atom (H) or a radical R₁;
Y is a divalent group;
R¹, R¹¹, R⁴, R¹⁴, R⁵ and R¹⁵ are independently a substituted or unsubstituted hydrocarbyl group;
R³ and R¹³ are independently a divalent group;
w and ww are independently integers ranging from 0 to 1000
x and xx are independently integers ranging from 1 to 100;
y and yy are independently integers ranging from 0 to 1000;
preferably, w and y are not simultaneously equal to 0 and ww and yy are not simultaneously equal to 0.

18. Composition according to claim 17, wherein. the silicone elastomer of formula (I) is selected from the following compounds 1 to 12:

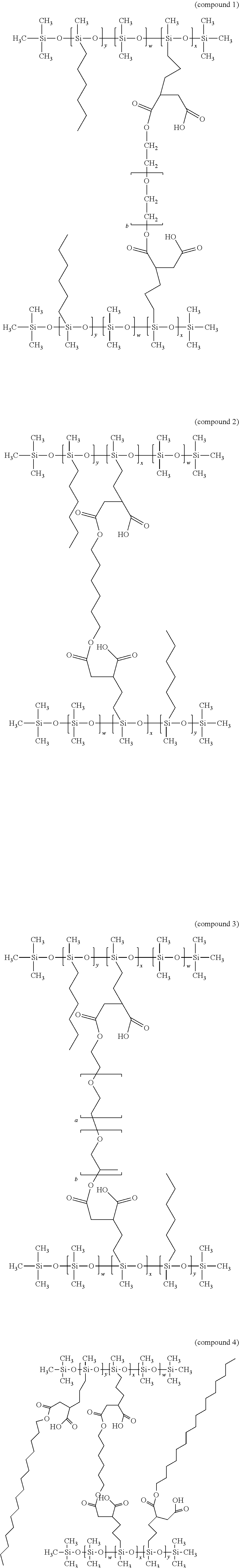

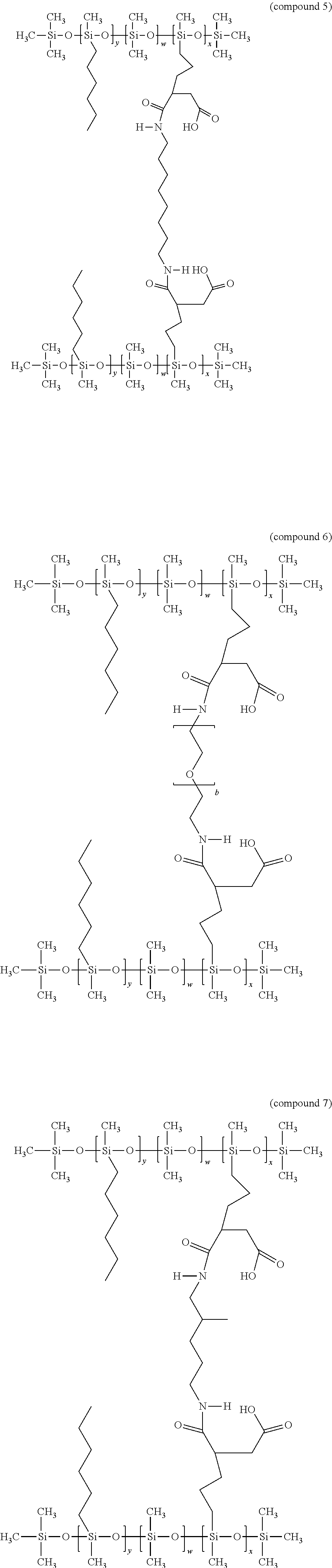

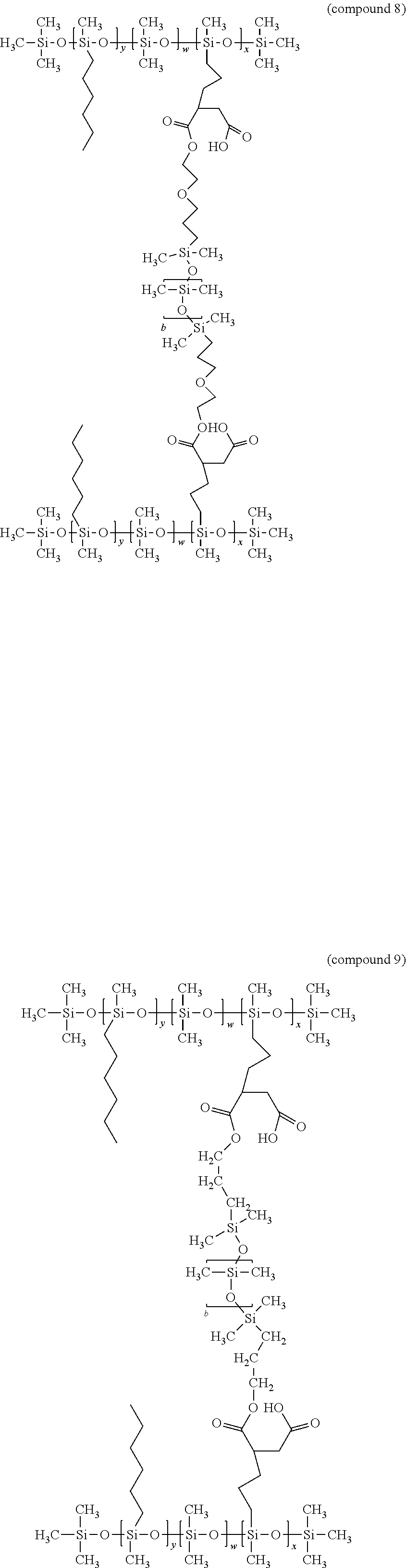

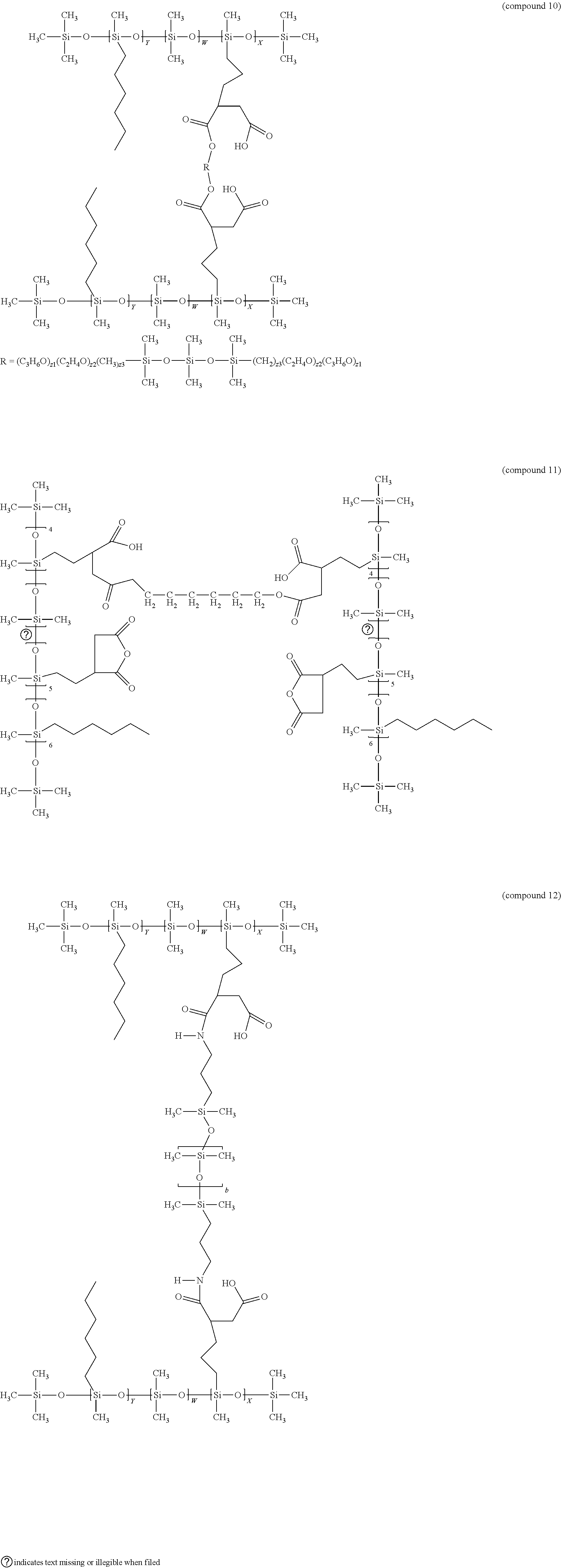

19. Composition according to claim 18, wherein the carboxylic acid functional silicone elastomer is compound 10 or compound 11.

20. Composition according to any one of claims 1 to 16, wherein the carboxylic acid functional silicone elastomer is that of INCI name: HEXYL/SUCCINYL DIMETHICONE CROSSPOLYMER.

21. Composition according to any one of the preceding claims, wherein the carboxylic acid-functional silicone elastomer is dispersed in at least one H₂ oil and in gel form; preferably said H2 oil is selected from volatile hydrocarbon oils, volatile silicone oils, nonvolatile hydrocarbon oils, nonvolatile silicone oils, and mixtures thereof.

22. Composition according to claim 21, wherein the H₂ oil is a volatile hydrocarbon oil selected from those defined for H₁ oil in any of claims 1 to 4.

23. Composition according to claim 21 or 22, wherein the H₂ oil and the H₁ oil are identical, in particular denote isododecane.

24. Composition according to any one of claims 21 to 23, wherein the carboxylic acid-functional silicone elastomer oil dispersion comprises from 10 to 40% by weight of silicone elastomer active material, and more preferably from 20 to 35% by weight based on the total weight of the oil dispersion.

25. Composition according to any one of claims 21 to 24 wherein the carboxylic acid functional silicone elastomer is dispersed in isododecane and in gel form.

26. Composition according to claim 19, wherein the carboxylic acid-functional silicone elastomer is compound 10 or compound 11 and present in the isododecane-based dispersion at an active content of 30% by weight.

27. Composition according to claim 20, comprising at least the silicone elastomer in gel form dispersed in isododecane having the INCI name: ISODODECANE (and) HEXYL/SUCCINYL DIMETHICONE CROSSPOLYMER, and more particularly at an active material content of 30% by weight in the isododecane-based dispersion.

28. Composition according to any one of the preceding claims, further comprising at least one silicone resin and/or at least one dimethiconol silicone-silicone copolymer.

29. Composition according to claim 28, wherein the silicone resin is an MQ resin, more particularly a Trimethylsiloxysilicate resin.

30. Composition according to claim 28, wherein the dimethiconol type silicone/silicone resin copolymer is a copolymer with the INCI name: TRIMETHYLSILOXYSILICATE/DIMETHICONOL CROSSPOLYMER.

31. Composition according to any one of the preceding claims, additionally comprising at least one silicone polyamide, in particular with the INCI name: NYLON-611/DIMETHICONE COPOLYMER.

32. Composition according to any one of the preceding claims, further comprising at least one pulverulent colorant, preferably an inorganic pigment, in particular said inorganic pigment comprises a lipophilic or hydrophobic coating.

33. Composition according to claim 32, wherein the pulverulent colorant(s) is (are) present, in a content ranging from 5 to 40 wt %, preferably from 10 wt % to 30 wt %, more preferably from 15 to 25 wt % based on the total weight of the composition.

34. Process for coating keratinous materials, more particularly for making up and/or caring for keratinous materials, such as the skin, characterized in that it comprises the application to the keratinous materials of a composition as defined in any one of the preceding claims.
